SEQUENCE LISTING (4 sequences)

SEQ ID NO: 1 - DNA, 21 bp, Artificial Sequence
  Synthetic polynucleotide
  misc_feature (4)..(4): 2′-O-methyl modified ribonucleotide
  misc_feature (6)..(6): 2′-O-methyl modified ribonucleotide
  misc_feature (8)..(12): 2′-O-methyl modified ribonucleotide
  misc_feature (15)..(16): 2′-O-methyl modified ribonucleotide
  misc_feature (18)..(18): 2′-O-methyl modified ribonucleotide
  misc_feature (20)..(21): DNA overhang
ggauaugcccuugacuauatt

SEQ ID NO: 2 - DNA, 21 bp, Artificial Sequence
  Synthetic polynucleotide
  misc_feature (3)..(3): 2′-O-methyl modified ribonucleotide
  misc_feature (7)..(7): 2′-O-methyl modified ribonucleotide
  misc_feature (13)..(13): 2′-O-methyl modified ribonucleotide
  misc_feature (15)..(15): 2′-O-methyl modified ribonucleotide
  misc_feature (20)..(21): DNA overhang
uauagucaagggcauaucctt

SEQ ID NO: 3 - DNA, 21 bp, Artificial Sequence
  Synthetic polynucleotide
  misc_feature (3)..(3): 2′-O-methyl modified ribonucleotide
  misc_feature (6)..(11): 2′-O-methyl modified ribonucleotide
  misc_feature (14)..(17): 2′-O-methyl modified ribonucleotide
  misc_feature (20)..(21): DNA overhang
gaugaucucucaacuuuaatt

SEQ ID NO: 4 - DNA, 21 bp, Artificial Sequence
  Synthetic polynucleotide
  misc_feature (2)..(2): 2′-O-methyl modified ribonucleotide
  misc_feature (16)..(16): 2′-O-methyl modified ribonucleotide
  misc_feature (20)..(21): DNA overhang
uuaaaguugagagaucauctt

(19) United States
(12) Patent Application Publication
(10) Pub. No.: US 2023/0062603 A1
(43) Pub. Date: Mar. 2, 2023

(54) FORMULATIONS FOR DELIVERY OF OLIGONUCLEOTIDES TO LUNG CELLS

(71) Applicant: Translate Bio MA, Inc., Lexington, MA (US)

(72) Inventors: Balkrishen Bhat, Carlsbad, CA (US); Brian Bettencourt, Groton, MA (US); Saswata Karmakar, Acton, MA (US); Caroline Woo, Newton, MA (US); John Androsavich, Lexington, MA (US); Shrirang Karve, Lexington, MA (US)

(73) Assignee: Translate Bio MA, Inc., Lexington, MA (US)

(21) Appl. No.: 17/791,190

(22) PCT Filed: Jan. 6, 2021
(86) PCT No.: PCT/US2021/012251

§ 371 Date: Jul. 6, 2022

Related U.S. Application Data
(60) Provisional application No. 62/958,034, filed on Jan. 7, 2020.

(51) Int. Cl. A61K 31/7088; A61K 9/00; A61K 9/127; C12N 15/113; C12N 15/88

(52) U.S. Cl. CPC A61K 31/7088; A61K 9/0078; A61K 9/1272; C12N 15/1137; C12N 15/88; C12N 2310/14; C12N 2320/32

(57) ABSTRACT

The present disclosure provides a method of pulmonarily administering an oligonucleotide to lung cells. In some embodiments, the disclosure provides formulations that comprise an oligonucleotide that is complementary to a target gene in lung cells and a lipid nanoparticle. Methods of the present disclosure are useful in modifying expression of target genes associated with chronic obstructive pulmonary disease, asthma, and pulmonary fibrosis.

### CROSS-REFERENCE TO RELATED APPLICATIONS

This application claims the benefit under 35 U.S.C. § 119(e) of U.S. Provisional Application No. U.S. 62/958,034, filed on Jan. 7, 2020, the contents of which are incorporated herein by reference in their entirety.

### FIELD OF THE INVENTION

The disclosure relates to formulations, e.g., nanoparticle formulations, as well as methods of using such formulations for delivering oligonucleotides.

### REFERENCE TO THE SEQUENCE LISTING

The present application is being filed along with a Sequence Listing in electronic format. The Sequence Listing is provided as a file entitled R0693.70055W000-SEQ.txt created on Jan. 5, 2021 which is 2 kilobytes in size. The information in electronic format of the sequence listing is incorporated herein by reference in its entirety.

### BACKGROUND OF THE INVENTION

A considerable portion of human diseases of the lung are associated with aberrant expression of a single or a few genes. Some of these diseases, including chronic obstructive pulmonary disease, asthma, and pulmonary fibrosis, have increased expression of a mutated protein, over-expression of a wild-type protein, or a combination thereof. Current methods of treating lung disease associated with aberrant gene expression are limited because of the difficulty in delivering oligonucleotide based therapeutics directly to lung cells.

### SUMMARY OF THE INVENTION

Formulations provided herein are useful for delivering oligonucleotides to the lung by pulmonary delivery, e.g., nebulization. In some embodiments, these formulations advantageously do not substantially induce an immune response. In some embodiments, these immune-evading formulations result in improved delivery because they minimize inflammatory responses.

In one aspect, provided herein is a method of delivering an oligonucleotide to lung cells of a subject, the method comprising pulmonarily administering to the subject an aerosolized formulation that comprises i) an oligonucleotide comprising an anti sense strand having a length of 8-30 nucleotides and having a region of complementarity with a target gene in the lung cells, wherein the oligonucleotide comprises at least one modified nucleotide, and ii) a lipid nanoparticle.

In another aspect, provided herein is formulation comprising an oligonucleotide having at least one modified nucleotide and comprising an antisense strand of 8-30 nucleotides in length; and a lipid nanoparticle, wherein the antisense strand has a region of complementarity with a target gene, and wherein the composition is formulated for delivery to the lung.

In another aspect, provided herein is a kit comprising a container housing the any of the formulations described herein.

In some embodiments, the formulation is an immune-evading formulation.

In some embodiments, the lipid nanoparticle comprises one or more cationic lipids, one or more non-cationic lipids, one or more PEG-modified lipids, or a combination thereof. In some embodiments, the lipid nanoparticle comprises a cationic lipid selected from DOTAP (1,2-dioleyl-3-trimethylammonium propane), DODAP (1,2-dioleyl-3-dimethylammonium propane), DOTMA (N-[1-(2,3-dioleyloxy)propyl]-N,N,N-trimethylammonium chloride), DLinKC2DMA, DLin-KC2-DM, C12-200, cKK-E12 (3,6-bis(4-(bis(2-hydroxydodecyl)amino)butyl)piperazine-2,5-dione), HGT5000, HGT5001, HGT4003, ICE, OF-02 and combinations thereof. In some embodiments, the lipid nanoparticle comprises a non-cationic lipid selected from DSPC (1,2-distearoyl-sn-glycero-3-phosphocholine), DPPC (1,2-dipalmitoyl-sn-glycero-3-phosphocholine), DOPE (1,2-dioleyl-sn-glycero-3-phosphoethanolamine), DOPC (1,2-dioleyl-sn-glycero-3-phosphotidylcholine) DPPE (1,2-dipalmitoyl-sn-glycero-3-phosphoethanolamine), DMPE (1,2-dimyristoyl-sn-glycero-3-phosphoethanolamine), DOPG (1,2-dioleoyl-sn-glycero-3-phospho-(1′-rac-glycerol)) or combinations thereof. In some embodiments, the one or more PEG-modified lipids are conjugated lipids that inhibits aggregation of particles. In some embodiments, the lipid nanoparticle comprises ICE, DOPE and DMG-PEG2K.

In some embodiments, the cationic lipid constitutes about 30-80% of the lipid nanoparticles by molar ratio. In some embodiments, the ratio of cationic lipids:non-cationic lipids:PEGylated lipids is approximately 60:45:5 by molar ratio.

In some embodiments, the lipid nanoparticle has a size of about 80 nm to 120 nm as measured by dynamic light scattering.

In some embodiments, the oligonucleotide is encapsulated in the lipid nanoparticle.

In some embodiments, providing the formulation to the lung of the subject is by nebulization. In some embodiments, the lung cells are lung epithelial cells. In some embodiments, the subject is identified as being at risk of lung disease, e.g., COPD, asthma, or pulmonary fibrosis.

In some embodiments, the region of complementarity comprises at least 15 contiguous nucleotides of the target gene. In some embodiments, the oligonucleotide is single stranded. In some embodiments, the oligonucleotide further comprises a sense strand, and wherein the antisense strand and the sense strand form a duplex region. In some embodiments, the sense strand and/or the antisense strand is 15-25 nucleotides in length. In some embodiments, the region of complementarity is 19 nucleotides in length. In some embodiments, the oligonucleotide comprising the sense strand and the antisense strand and having a duplex region further comprises a single-stranded overhang on the sense and/or antisense strand in the range of 1 to 2 nucleotides in length. In some embodiments, the sense strand and/or the antisense strand have a 3′ overhang comprising two deoxythymidines. In some embodiments, the oligonucleotide comprises one blunt end. In some embodiments, the nucleotide at the 5′ end of the sense strand and/or antisense strand is uracil.

In some embodiments, the modified nucleotide comprises a 2′-fluoro or a 2′-O-methyl. In some embodiments, the oligonucleotide comprises 2′-O-methyl modified nucleotides in both the sense strand and the antisense strand. In some embodiments, the oligonucleotide comprises more 2′-O-methyl modified nucleotides on the sense strand than on the antisense strand.

### BRIEF DESCRIPTION OF THE FIGURES

FIG. 1 illustrates relative HPRT1 knockdown after PC9 cells are treated with siRNA duplexes targeting HPRT1 (#2 or #3) relative to control (cells treated with lipid nanoparticle only). HPRT1 expression was normalized to FXN (non-target gene) expression.

FIG. 2 illustrates relative HPRT1 knockdown after PC9 cells are treated with siRNA duplex #2 and transfection agent (Dharmafect) or after PC9 cells are treated with a composition comprising siRNA duplex #2 and a lipid nanoparticle as in the present disclosure relative to control (cells treated with lipid nanoparticle only). HPRT1 expression was normalized to FXN (non-target gene) expression.

FIG. 3 illustrates HPRT1 knockdown in the liver. Compositions comprising MC3 lipid nanoparticle and siRNA duplex #2 knocked down HPRT1 expression relative to control (saline) at almost all siRNA doses (0.1, 0.25, and 0.5 mg/kg) and time points measured (24 and 72 hours). Compositions comprising C12-200 lipid nanoparticle and siRNA duplex #2 knocked down HPRT1 expression relative to control (saline) at all siRNA doses (0.1, 0.25, 0.5 mg/kg) and time points measured (24 and 72 hours).

FIG. 4 illustrates HPRT1 knockdown in the lung. Compositions comprising siRNA duplex #2 and lipid nanoparticle show statistically significant HPRT1 knockdown of up to 30% in the lungs after single and double treatments with the compositions. HPRT1 gene expression was normalized to PPIB and GUSB (non-target genes) and compared to mice treated with control compositions (saline).

### DETAILED DESCRIPTION OF THE INVENTION

According to some aspects, the disclosure provides formulations comprising oligonucleotides and lipid nanoparticles, and methods of use in pulmonary delivery, e.g., nebulization. In some embodiments, immune-evading formulations are provided that result in improved oligonucleotide delivery to lung cells. In some embodiments, the lipid nanoparticles of the formulations comprise one or more cationic lipids, one or more non-cationic lipids (e.g., anionic or neutral), one or more PEG-modified lipids, or a combination thereof. The oligonucleotides provided herein are configured to modulate expression of target genes in cells of the lung. In some embodiments, methods and formulations provided herein are useful in subject having or suspected of having a lung disease (e.g., chronic obstructive pulmonary disease, asthma, pulmonary fibrosis, etc.).

Further aspects of the disclosure, including a description of defined terms, are provided below.

### I. DEFINITIONS

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Immune-evading formulation: As is used herein, “immune-evading formulation,” with respect to oligonucleotides, refers to an oligonucleotide formulation that does not substantially induce an immune response following administration (e.g., via pulmonary administration) to a subject, having an otherwise competent immune system relative, compared to administration of an appropriate control formulation (e.g., a naked oligonucleotide formulation) to an appropriate control subject. In some embodiments, an immune-evading formulation does not substantially induce inflammation, alteration of immune-related cytokine and/or chemokine levels, effector T-cell proliferation, and/or B-cell proliferation. In some embodiments, an immune-evading formulation is not substantially targeted by (i) the mucociliary clearance system, (ii) alveolar macrophages, and/or (iii) monocytes. In some embodiments, an immune-evading formulation does not produce a detectable immune response, e.g, inflammation or targeting by (i) the mucociliary clearance system, (ii) alveolar macrophages, and/or (iii) monocytes, (e.g., measuring cytokine and chemokine levels, measuring T-cell proliferation, measuring B-cell proliferation), or a less than a 10% increase in immune response relative to an appropriate control (e.g., a naked oligonucleotide), e.g., 0-10%, 1, 2, 3, 4, 5, 6, 7, 8, 9, or 10% increase in immune response.

Lung cells: As used herein, the term “lung cells” refers to any cells that reside in the lungs. In some embodiments, lung cells are cells that carry out functions relevant to lung function, development, regeneration and/or repair. In some embodiments, lung cells arise from cells forming the lung endoderm. Non-limiting examples of lung cells include: epithelial cells, type I alveolar cells, type II alveolar cells, macrophages, fibroblasts, neuroendocrine cells, basal cells, secretory cells, ciliated cells, Clara cells, and endothelial cells. In some embodiments, lung cells are epithelial cells residing in the secondary bronchi, tertiary bronchi, bronchioles, and/or alveoli regions of the lung.

Subject: As used herein, the term “subject” refers to a human or any non-human animal (e.g., mouse, rat, rabbit, dog, cat, cattle, swine, sheep, horse or primate). A subject can be a patient, which refers to a subject, e.g., a human, presenting to a medical or health care provider for evaluation and/or treatment, including for diagnosis or treatment of a lung disease. A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

Complementary: refers to the capacity for precise base pairing between two nucleotides. For example, if a nucleotide at a certain position of an oligonucleotide is capable of hydrogen bonding with a nucleotide at the same position of a target sequence as described herein, then the oligonucleotide and the target sequence are considered to be complementary to each other at that position. An oligonucleotide and target sequence are complementary to each other when a sufficient number of corresponding positions in each molecule are occupied by nucleotides that can hydrogen bond with each other through their bases. With respect of an oligonucleotide the term complementary may be used to indicate a sufficient degree of complementarity or precise base pairing such that stable and specific binding occurs between the oligonucleotide and a target nucleic acid (e.g., between an antisense strand of a oligonucleotide and an mRNA). Thus, it should be appreciated 100% complementarity is not required for an oligonucleotide to be consider complementary to a target sequence provided that a sufficient degree of complementarity or precise base pairing exist to achieve stable and specific binding between the oligonucleotide and target nucleic acid.

Oligonucleotide: As used herein, the term “oligonucleotide” refers to a single stranded or double stranded nucleic acid of 100 nucleotides or less. In some embodiments, the oligonucleotide is single stranded and comprises a sense or antisense strand. In some embodiments, the oligonucleotide has a region of complementarity to a target gene. In some embodiments, the oligonucleotide is an siRNA. In some embodiments, the oligonucleotide is an antisense oligonucleotide. In some embodiments, oligonucleotides are classified as deoxyribooligonucleotides or ribooligonucleotides. In some embodiments, oligonucleotides encompasses other nucleobase containing polymers, such as, without limitation, peptide nucleic acids (PNA), peptide nucleic acids with phosphate groups (PHONA), locked nucleic acids (LNA), morpholino-backbone oligonucleotides and oligonucleotides having backbone sections with alkyl linkers or amino linkers. Oligonucleotides also include naturally occurring nucleotides, modified nucleotides, e.g., modified nucleotides described herein, or mixtures thereof.

Cationic Lipids: As used herein, the term “cationic lipids” refers to lipid and lipidoid molecules or moieties that have a net positive charge at a selected pH, such as at a physiological pH or a pH in the range of 7.0 to 7.4. Suitable cationic lipids for use in the compositions and methods provided herein include the cationic lipids as described in International Patent Publication WO 2010/144740, which is incorporated herein by reference, and elsewhere in this disclosure.

Administering: As used herein, the term “administering” refers to bringing a patient, tissue, organ or cells in contact with the formulation described herein. As used herein, administration can be accomplished in vitro, i.e. in a test tube, or in vivo, i.e. in cells or tissues of living organisms, for example, humans. In certain embodiments, the present invention encompasses administering the compounds useful in the present invention to a patient or subject. In some embodiments, administering comprises administering to lung cells. In some embodiments, administering comprises pulmonarily administering.

Pulmonarily administering: As used herein, the term “pulmonarily administering” refers to administering the formulation described herein to lung cells in vivo by delivering the formulation to the lung. Non-limiting methods of pulmonary delivery include: nebulization, atomization, intratracheal administration, and intratracheal instillation.

Aerosolized formulation: As used herein, the term “aerosolized formulation” refers to a mixture of liquid (e.g., liquid droplets) or particles and air or other inhalable gas. In some embodiments, an aerosolized formulation comprises a fine spray or dispersed suspension that can be inhaled. In some embodiments, aerosolization is accomplished using a propellant or other suitable energy source (e.g., ultrasound energy) to convert liquid or particles into a fine spray or dispersed suspension.

Lipid nanoparticle: As used herein, the term “lipid nanoparticle” refers to a discrete object comprised of one or more lipids and possessing at least one dimension that is generally less than or equal to 5 micron in size. Lipid nanoparticles may be a variety of different shapes, including but not limited to spheroidal, cuboidal, pyramidal, oblong, cylindrical, toroidal, and the like. In some formulations, lipid nanoparticle comprise at least one cationic lipid, at least one non-cationic lipid, and at least one aggregation prevention lipid, e.g., PEG-modified lipid.

PEG-modified lipid: As used herein, the term “PEG-modified lipid” refers to a lipid comprising one or more polyethylene glycol molecules. In some embodiments, the one or more polyethylene glycol molecules are covalently attached to the lipid.

### II. INTRODUCTION

The present disclosure provides a method of delivering an oligonucleotide to lung cells of a subject, the method comprising pulmonarily administering to the subject an aerosolized formulation that comprises i) an oligonucleotide comprising an antisense strand having a length of 8-30 nucleotides and having a region of complementarity with a target gene in the lung cells, wherein the oligonucleotide comprises at least one modified nucleotide, and ii) a lipid nanoparticle.

The formulation advantageously pulmonarily delivered to the cells of the lung. For example, in some embodiments, the formulation is pulmonarily delivered to lung epithelial cells, e.g., lung epithelial cells residing in the secondary bronchi, tertiary bronchi, bronchioles, and/or alveoli regions of the lung.

In some embodiments, the formulation administered to the lung is immune-evading. Immune-evading formulations may, for example, evade an inflammatory response, thereby increasing efficacy of delivery of the formulation.

The formulation comprises an oligonucleotide and a lipid nanoparticle. Oligonucleotides of the present disclosure have a region of complementarity with a target gene in lung cells. The oligonucleotides may be single-stranded, double-stranded, or double-stranded with single-stranded overhang(s). The oligonucleotides can be, for example, siRNA or antisense oligonucleotides.

In some embodiments, lipid nanoparticles are composed of cationic lipids, anionic lipids, and aggregation-prevention lipids. The oligonucleotides of the present disclosure can be encapsulated within the nanoparticle.

### III. OLIGONUCLEOTIDES

The present disclosure provides an oligonucleotide comprising an antisense strand and/or a sense strand. An oligonucleotide in the present disclosure may be a siRNA, shRNA, miRNA, amiRNA, or an antisense oligonucleotide (ASO). Oligonucleotides described herein can be formulated with a particle for administration by pulmonary delivery to a subject for treating a condition associated with aberrant expression of a target gene. Aberrant expression of a target gene may be either increased expression of a target gene, decreased expression of a target gene, or expression of a mutated form of a target gene that is associated with a condition. It should be understood that the formulations, compositions and methods can be practiced with any of the oligonucleotides disclosed herein.

The present disclosure provides a formulation comprising an oligonucleotide comprising an antisense strand of 8-30 nucleotides in length. An antisense strand is complementary to and forms a duplex region with the target gene.

The oligonucleotide of the present disclosure may be composed of a ribonucleotide, a deoxyribonucleotide, and/or a bridged nucleotide. In some embodiments, the oligonucleotide comprises at least 1 ribonucleotide, at least 1 deoxyribonucleotide, or at least 1 bridged nucleotide.

The oligonucleotide may further comprise a sense strand, wherein the antisense strand and the sense strand form a duplex region. In some embodiments, the sense and/or the antisense strand is 15-25 nucleotides in length. In some embodiments, the sense and/or the antisense strand is 8-30 nucleotides in length. In some embodiments, the sense and/or the antisense strand is 10-30 nucleotides in length. In some embodiments, the sense and/or the antisense strand is 8, 9, 10, 11, 12, 13, 14, 15, 16, 17, 18, 19, 20, 21, 22, 23, 24, 25, 26, 27, 28, 29, or 30 nucleotides in length.

An oligonucleotide of the present disclosure may be either single stranded or double stranded. Double stranded oligonucleotides have a duplex region that is at least 1 nucleotide long. In some embodiments, the duplex region is 1-40 nucleotides long. In some embodiments, the duplex region is 10-30 nucleotides long. In some embodiments, the duplex region is 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides long.

In some embodiments, the oligonucleotide comprises an antisense strand that is complementary to a target gene. The antisense strand may be at least 80% complementary to (optionally one of at least 85%, 90%, 91%, 92%, 93%, 94%, 95%, 96%, 97%, 98%, 99% or 100% complementary to) the consecutive nucleotides of a target gene. In some embodiments an antisense strand may contain 1, 2 or 3 base mismatches compared to the portion of the consecutive nucleotides of the target gene. In some embodiments an antisense strand may have up to 3 mismatches over 15 bases, or up to 2 mismatches over 10 bases. In some embodiments, the complementarity is over the full length of the antisense strand. In some embodiments, the complementarity extends over a portion of the antisense strand.

In some embodiments, the antisense strand have a region of complementarity with a target gene transcript that has less than a threshold level of complementarity with every sequence of nucleotides, of equivalent length, of an off-target gene. For example, the antisense strand may be designed to ensure that it does not have a sequence that targets genes in a cell other than the target gene. The threshold level of sequence identity may be 50%, 60%, 70%, 80%, 85%, 90%, 95%, 99% or 100% sequence identity.

In some embodiments, the antisense strand may be complementary to target genes encoded by homologues of a gene across different species (e.g., a mouse, rat, rabbit, goat, monkey, etc.) In some embodiments, antisense strands having these characteristics may be tested in vivo or in vitro for efficacy in multiple species (e.g., human and mouse). This approach also facilitates development of clinical candidates for treating human disease by selecting a species in which an appropriate animal exists for the disease.

In some embodiments, the region of complementarity of an antisense strand is complementary with at least 8 to 15, 8 to 30, 8 to 40, or 10 to 50, or 5 to 50, or 5 to 40 bases, e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 consecutive nucleotides of a target gene. In some embodiments, the region of complementarity is complementary with at least 8 consecutive nucleotides of a target gene.

Complementarity does not require 100% base-pairing between the antisense strand and the target gene. In some embodiments, the antisense strand comprises a region that is 100% complementary to a region in the target gene. In some embodiments, the antisense strand comprises a region that is at least 90% complementary to a region in the target gene. In some embodiments, the antisense strand comprises a region that is at least 80% complementary to a region in the target gene. In some embodiments, the antisense strand comprises a region that is at least 70% complementary to a region in the target gene. In some embodiments, the antisense strand comprises a region that is at least 60% complementary to a region in the target gene. In some embodiments, the antisense strand comprises a region that is at least 50% complementary to a region in the target gene.

In the present disclosure, the region of complementarity between the antisense nucleotide and the target gene comprises at least 15 contiguous nucleotides of the target gene. In some embodiments, the region of complementarity between the antisense oligonucleotide and the target gene comprises 8-30 contiguous nucleotides of the target gene. In some embodiments, the region of complementarity between the anti sense oligonucleotide and the target gene comprises 15-30 contiguous nucleotides of the target gene. In some embodiments, the region of complementarity between the anti sense oligonucleotide and target gene comprises at least 8, 9, 10, 11, 12, 13, 14, 15, 16, 17, 18, 19, 20, 21, 22, 23, 24, 25, 26, 27, 28, 29, or 30 contiguous nucleotides of the target gene.

In some embodiments, a complementary nucleic acid sequence need not be 100% complementary to that of its target to be specifically hybridizable. In some embodiments, an antisense strand for purposes of the present disclosure is specifically hybridizable with a target gene when hybridization of the oligonucleotide to the target gene promotes degradation of the target gene, and when there is a sufficient degree of complementarity to avoid non-specific binding of the sequence to non-target sequences under conditions in which avoidance of non-specific binding is desired, e.g., under physiological conditions in the case of in vivo assays or therapeutic treatment, and in the case of in vitro assays, under conditions in which the assays are performed under suitable conditions of stringency.

Base pairings may include both canonical Watson-Crick base pairing and non-Watson-Crick base pairing (e.g., Wobble base pairing and Hoogsteen base pairing). It is understood that for complementary base pairings, adenosine-type bases (A) are complementary to thymidine-type bases (T) or uracil-type bases (U), that cytosine-type bases (C) are complementary to guanosine-type bases (G), and that universal bases such as 3-nitropyrrole or 5-nitroindole can hybridize to and are considered complementary to any A, C, U, or T. Inosine (I) has also been considered in the art to be a universal base and is considered complementary to any A, C, U or T.

In some embodiments, any one or more thymidine (T) nucleotides (or modified nucleotide thereof) or uridine (U) nucleotides (or a modified nucleotide thereof) in a sequence provided herein, including a sequence provided in the sequence listing, may be replaced with any other nucleotide suitable for base pairing (e.g., via a Watson-Crick base pair) with an adenosine nucleotide. In some embodiments, any one or more thymidine (T) nucleotides (or modified nucleotide thereof) or uridine (U) nucleotides (or a modified nucleotide thereof) in a sequence provided herein, including a sequence provided in the sequence listing, may be suitably replaced with a different pyrimidine nucleotide or vice versa. In some embodiments, any one or more thymidine (T) nucleotides (or modified nucleotide thereof) in a sequence provided herein, including a sequence provided in the sequence listing, may be suitably replaced with a uridine (U) nucleotide (or a modified nucleotide thereof) or vice versa. In some embodiments, the nucleotide at an end of the sense and/or antisense strand is uracil. In some embodiments, the nucleotide at the 5′ end of the sense and/or antisense strand is uracil. In some embodiments, the nucleotide at the 3′ end of the sense and/or antisense strand is uracil.

In some embodiments, the oligonucleotide comprising the sense and/or the antisense strand having a duplex region further comprises at least one single-stranded overhang. A single-stranded overhang is an oligonucleotide region outside of the duplex region that is not complementary the sense or antisense strand. In some embodiments, both the sense and the antisense strand have at least one single-stranded overhang. In some embodiments, the sense and/or the anti sense strand have a 5′ single-stranded overhang. In some embodiments, the sense and/or the antisense strand have a 3′ single-stranded overhang. In some embodiments, the sense and/or the antisense strand have a 5′ single-stranded overhang and a 3′ single-stranded overhang.

In some embodiments, a single-stranded overhang is at least 1 nucleotide. In some embodiments, a single-stranded overhang is less than 29 nucleotides. In some embodiments, a single-stranded overhang is 2-28 nucleotides in length. In some embodiments, a single-stranded overhang that is 1, 2, 3, 4, 5, 6, 7, 8, 9, 10, 11, 12, 13, 14, 15, 16, 17, 18, 19, 20, 21, 22, 23, 24, 25, 26, 27, 28, or 29 nucleotides in length.

The sense and/or antisense strand may have a 3′ single-stranded overhang comprising at least 1 deoxythymidines (dTs). In some embodiments, the sense and/or antisense strand may have a 3′ single-stranded overhang comprising 8 deoxythymidines. In some embodiments, the sense and/or antisense strand may have a single-stranded overhang comprising 1-10 deoxythymidines. In some embodiments, the sense and/or antisense strand have a 3′ single-stranded overhang comprising 1, 2, 3, 4, 5, 6, 7, 8, 9, 10, 11, or 12 deoxythymidines.

The sense and/or antisense strand may comprise at least 1 blunt end. A blunt end refers to a sense and/or antisense strand that forms a duplex region at least 1 end. In some embodiments, the sense and/or antisense strand comprises a 5′ blunt end. In some embodiments, the sense and/or antisense strand comprises a 3′ blunt end. In some embodiments, the sense and/or antisense strand comprises a 5′ blunt end and a 3′ blunt end.

In some embodiments, an oligonucleotide may have a sequence that does not contain guanosine nucleotide stretches (e.g., 3 or more, 4 or more, 5 or more, 6 or more consecutive guanosine nucleotides). In some embodiments, oligonucleotides having guanosine nucleotide stretches have increased non-specific binding and/or off-target effects, compared with oligonucleotides that do not have guanosine nucleotide stretches. Contiguous runs of three or more Gs or Cs may not be preferable in some embodiments. Accordingly, in some embodiments, the oligonucleotide does not comprise a stretch of three or more guanosine nucleotides.

An oligonucleotide may have a sequence that is has greater than 30% G-C content, greater than 40% G-C content, greater than 50% G-C content, greater than 60% G-C content, greater than 70% G-C content, or greater than 80% G-C content. An oligonucleotide may have a sequence that has up to 100% G-C content, up to 95% G-C content, up to 90% G-C content, or up to 80% G-C content. In some embodiments, GC content of an oligonucleotide is preferably between about 30-60%.

It is to be understood that any oligonucleotide provided herein can be excluded.

In some embodiments, it has been found that oligonucleotides disclosed herein may modulate the expression of a target gene by at least about 50% (e.g. 150% of normal or 1.5 fold), or by about 2 fold to about 5 fold. In some embodiments, target gene expression may be decreased by at least about 1.5 fold, 2 fold, 5 fold, 10 fold, 15 fold, 20 fold, 30 fold, 40 fold, 50 fold, or 100 fold, or any range between any of the foregoing numbers. In some embodiments, target gene expression may be increased by at least about 1.5 fold, 2 fold, 5 fold, 10 fold, 15 fold, 20 fold, 30 fold, 40 fold, 50 fold or 100 fold, or any range between any of the foregoing numbers. In some embodiments, decreased target gene expression has been shown to correlate to decreased protein expression. In some embodiments, increased target gene expression has been shown to correlate to increased protein expression.

It is understood that any reference to uses of oligonucleotides or other molecules throughout the description contemplates use of the oligonucleotides or other molecules in preparation of a pharmaceutical composition or medicament for use in the treatment of condition or a disease associated with aberrant expression of a target gene. Thus, as one nonlimiting example, this aspect of the disclosure includes use of oligonucleotides or other molecules in the preparation of a medicament for use in the treatment of disease, wherein the treatment involves post transcriptionally altering protein and/or RNA levels in a targeted manner.

It should be appreciated the formulations disclosed herein can, in some embodiments, include nucleic acids of a variety of different formats, including nucleic acids (e.g., oligonucleotides) for directing gene editing enzymes, e.g., Cas9 and similar enzymes. In some embodiments, a formulation provided herein comprises a nucleic acid (e.g., an oligonucleotide) having a guide strand (e.g., a guide RNA) for directing gene editing enzymes and) a lipid nanoparticle.

a. Modifications

In some embodiments, it may be advantageous to synthesize an oligonucleotide of the present disclosure with one or more modified nucleotides. Typically, oligonucleotides are modified to enhance their stability or reduce their immunogenic properties, in particular when administered to a subject as naked oligonucleotides or in complexed form. Therefore, providing an oligonucleotide of the present disclosure may have synergistic effects, resulting in the induction of immune tolerance that exceeds what has been observed with unmodified oligonucleotides.

In some embodiments, oligonucleotides of the present disclosure are modified. Modifications of oligonucleotides can include, for example, modifications of the nucleotides. A modified oligonucleotide according to the disclosure can thus include, for example, backbone modifications, sugar modifications or base modifications. In some embodiments, oligonucleotides may be synthesized from naturally occurring nucleotides and/or nucleotide analogues (modified nucleotides) including, but not limited to, purines (adenine (A), guanine (G)) or pyrimidines (thymine (T), cytosine (C), uracil (U)), and as modified nucleotides analogues or derivatives of purines and pyrimidines, such as e.g. 1-methyl-adenine, 2-methyl-adenine, 2-methylthio-N-6-isopentenyl-adenine, N6-methyl-adenine, N6-isopentenyl-adenine, 2-thio-cytosine, 3-methyl-cytosine, 4-acetyl-cytosine, 5-methyl-cytosine, 2,6-diaminopurine, 1-methyl-guanine, 2-methyl-guanine, 2,2-dimethyl-guanine, 7-methyl-guanine, inosine, 1-methyl-inosine, pseudouracil (5-uracil), dihydro-uracil, 2-thio-uracil, 4-thio-uracil, 5-carboxymethylaminomethyl-2-thio-uracil, 5-(carboxyhydroxymethyl)-uracil, 5-fluoro-uracil, 5-bromo-uracil, 5-carboxymethylaminomethyl-uracil, 5-methyl-2-thio-uracil, 5-methyl-uracil, N-uracil-5-oxyacetic acid methyl ester, 5-methylaminomethyl-uracil, 5-methoxyaminomethyl-2-thio-uracil, 5′-methoxycarbonylmethyl-uracil, 5-methoxy-uracil, uracil-5-oxyacetic acid methyl ester, uracil-5-oxyacetic acid (v), 1-methyl-pseudouracil, queosine, .beta.-D-mannosyl-queosine, wybutoxosine, and phosphoramidates, phosphorothioates, peptide nucleotides, methylphosphonates, 7-deazaguanosine, 5-methylcytosine and inosine. The preparation of such analogues is known to a person skilled in the art e.g. from the U.S. Pat. Nos. 4,373,071, 4,401,796, 4,415,732, 4,458,066, 4,500,707, 4,668,777, 4,973,679, 5,047,524, 5,132,418, 5,153,319, 5,262,530 and 5,700,642, the disclosures of which are incorporated by reference in their entirety.

In some embodiments, oligonucleotides of the present disclosure may contain backbone modifications. Typically, a backbone modification is a modification in which the phosphates of the backbone of the nucleotides contained in the oligonucleotide are modified chemically. Exemplary backbone modifications typically include, but are not limited to, modifications from the group consisting of methylphosphonates, methylphosphoramidates, phosphoramidates, phosphorothioates (e.g. cytidine 5′-O-(1-thiophosphate)), boranophosphates, positively charged guanidinium groups etc., which means by replacing the phosphodiester linkage by other anionic, cationic or neutral groups.

In some embodiments, oligonucleotides of the present disclosure may contain sugar modifications. A typical sugar modification is a chemical modification of the sugar of the nucleotides it contains including, but not limited to, sugar modifications chosen from the group consisting of 2′-deoxy-2′-fluoro-oligoribonucleotide (2′-fluoro-2′-deoxycytidine 5′-triphosphate, 2′-fluoro-2′-deoxyuridine 5′-triphosphate), 2′-deoxy-2′-deamine-oligoribonucleotide (2′-amino-2′-deoxycytidine 5′-triphosphate, 2′-amino-2′-deoxyuridine 5′-triphosphate), 2′-O-alkyl oligoribonucleotide, 2′-deoxy-2′-C-alkyloligoribonucleotide (2′-O-methylcytidine 5′-triphosphate, 2′-methyluridine 5′-triphosphate), 2′-C-alkyloligoribonucleotide, and isomers thereof (2′-aracytidine 5′-triphosphate, 2′-arauridine 5′-triphosphate), or azidotriphosphates (2′-azido-2′-deoxycytidine 5′-triphosphate, 2′-azido-2′-deoxyuridine 5′-triphosphate).

In some embodiments, oligonucleotides of the present disclosure may contain modifications of the bases of the nucleotides (base modifications). A modified nucleotide which contains a base modification is also called a base-modified nucleotide. Examples of such base-modified nucleotides include, but are not limited to, 2-amino-6-chloropurine riboside 5′-triphosphate, 2-aminoadenosine 5′-triphosphate, 2-thiocytidine 5′-triphosphate, 2-thiouridine 5′-triphosphate, 4-thiouridine 5′-triphosphate, 5-aminoallylcytidine 5′-triphosphate, 5-aminoallyluridine 5′-triphosphate, 5-bromocytidine 5′-triphosphate, 5-bromouridine 5′-triphosphate, 5-iodocytidine 5′-triphosphate, 5-iodouridine 5′-triphosphate, 5-methylcytidine 5′-triphosphate, 5-methyluridine 5′-triphosphate, 6-azacytidine 5′-triphosphate, 6-azauridine 5′-triphosphate, 6-chloropurine riboside 5′-triphosphate, 7-deazaadenosine 5′-triphosphate, 7-deazaguanosine 5′-triphosphate, 8-azaadenosine 5′-triphosphate, 8-azidoadenosine 5′-triphosphate, benzimidazole riboside 5′-triphosphate, N1-methyladenosine 5′-triphosphate, N1-methylguanosine 5′-triphosphate, N6-methyladenosine 5′-triphosphate, 06-methylguanosine 5′-triphosphate, pseudouridine 5′-triphosphate, puromycin 5′-triphosphate or xanthosine 5′-triphosphate.

In some embodiments, oligonucleotides are provided with chemistries suitable for delivery, hybridization and stability within cells to target and modulate target gene expression. In some embodiments, the oligonucleotides downregulate target gene expression. In some embodiments, the oligonucleotides upregulate target gene expression. Furthermore, in some embodiments, oligonucleotide chemistries are provided that are useful for controlling the pharmacokinetics, biodistribution, bioavailability and/or efficacy of the oligonucleotides. Accordingly, oligonucleotides described herein may be modified, e.g., comprise a modified sugar moiety, a modified internucleoside linkage, a modified nucleotide and/or combinations thereof. In addition, the oligonucleotides may exhibit one or more of the following properties: do not induce substantial cleavage or degradation of the target RNA, e.g., an RNA expressed from the same chromosomal locus as the target gene; do not cause substantially complete cleavage or degradation of the target RNA; do not activate the RNAse H pathway; do not activate RISC; do not recruit any Argonaute family protein; are not cleaved by Dicer; do not mediate alternative splicing; are not immune stimulatory; are nuclease resistant; have improved cell uptake compared to unmodified oligonucleotides; are not toxic to cells or mammals; and may have improved endosomal exit. Oligonucleotides that are designed to interact with RNA to modulate gene expression are a distinct subset of base sequences from those that are designed to bind a DNA target (e.g., are complementary to the underlying genomic DNA sequence from which the RNA is transcribed).

Any of the oligonucleotides disclosed herein may be linked to one or more other oligonucleotides disclosed herein by a linker, e.g., a cleavable linker.

Oligonucleotides of the disclosure can be stabilized against nucleolytic degradation such as by the incorporation of a modification, e.g., a nucleotide modification. For example, nucleic acid sequences of the disclosure include a phosphorothioate at least the first, second, or third internucleotide linkage at the 5′ or 3′ end of the nucleotide sequence. As another example, the nucleic acid sequence can include a 2′-modified nucleotide, e.g., a 2′-deoxy, 2′-deoxy-2′-fluoro, 2′-O-methyl, 2′-O-methoxyethyl (2′-O-MOE), 2′-O-aminopropyl (2′-O-AP), 2′-O-dimethylaminoethyl (2′-O-DMAOE), 2′-O-dimethylaminopropyl (2′-O-DMAP), 2′-O-dimethylaminoethyloxyethyl (2′-O-DMAEOE), or 2?-O—N-methylacetamido (2′-O-NMA). As another example, the nucleic acid sequence can include at least one 2′ methyl-modified nucleotide, and in some embodiments, all of the nucleotides include a 2′ methyl modification. In some embodiments, the nucleic acids are “locked,” e.g., comprise nucleic acid analogues in which the ribose ring is “locked” by a methylene bridge connecting the 2′-O atom and the 4′-C atom.

In some embodiments, modified oligonucleotides of the present disclosures comprise a 2′-fluoro or a 2′-O-methyl. In some embodiments, modified oligonucleotides comprise 2′-O-methyl modified nucleotides in the sense or antisense strand. In some embodiments, modified oligonucleotides comprise 2′-O-methyl modified nucleotides in both the sense and antisense strand.

In some embodiments, modified oligonucleotides of the present disclosure comprise more 2′-O-methyl modified nucleotides on the sense strand than on the antisense strand. In some embodiments, modified oligonucleotides comprise at least 1 more 2′-O-methyl modified nucleotides on the sense strand than on the antisense strand. In some embodiments, modified oligonucleotides comprise 1-10 more 2′-O-methyl modified nucleotides on the sense strand than on the antisense strand. In some embodiments, modified oligonucleotides comprise 1, 2, 3, 4, 5, 6, 7, 8, 9, or 10 more 2′-O-methyl modified nucleotides on the sense strand than on the antisense strand.

Any of the modified chemistries or formats of oligonucleotides described herein can be combined with each other, and that one, two, three, four, five, or more different types of modifications can be included within the same molecule.

In some embodiments, the oligonucleotide may comprise at least one ribonucleotide, at least one deoxyribonucleotide, and/or at least one bridged nucleotide. In some embodiments, the oligonucleotide may comprise a bridged nucleotide, such as a locked nucleic acid (LNA) nucleotide, a constrained ethyl (cEt) nucleotide, or an ethylene bridged nucleic acid (ENA) nucleotide. Examples of such nucleotides are disclosed herein and known in the art. In some embodiments, the oligonucleotide comprises a nucleotide analog disclosed in one of the following United States Patent or Patent Application Publications: U.S. Pat. Nos. 7,399,845, 7,741,457, 8,022,193, 7,569,686, 7,335,765, 7,314,923, 7,335,765, and 7,816,333, US 20110009471, the entire contents of each of which are incorporated herein by reference for all purposes. The oligonucleotide may have one or more 2′ O-methyl nucleotides. The oligonucleotide may consist entirely of 2′ O-methyl nucleotides.

Often an oligonucleotide has one or more nucleotide analogues. For example, an oligonucleotide may have at least one nucleotide analogue that results in an increase in T. of the oligonucleotide in a range of 1° C., 2° C., 3° C., 4° C., or 5° C. compared with an oligonucleotide that does not have the at least one nucleotide analogue. An oligonucleotide may have a plurality of nucleotide analogues that results in a total increase in T. of the oligonucleotide in a range of 2° C., 3° C., 4° C., 5° C., 6° C., 7° C., 8° C., 9° C., 10° C., 15° C., 20° C., 25° C., 30° C., 35° C., 40° C., 45° C. or more compared with an oligonucleotide that does not have the nucleotide analogue.

The oligonucleotide may be of up to 50 nucleotides in length in which 2 to 10, 2 to 15 2 to 16, 2 to 17, 2 to 18, 2 to 19, 2 to 20, 2 to 25, 2 to 30, 2 to 40, 2 to 45, or more nucleotides of the oligonucleotide are nucleotide analogues. The oligonucleotide may be of 8 to 30 nucleotides in length in which 2 to 10, 2 to 15 2 to 16, 2 to 17, 2 to 18, 2 to 19, 2 to 20, 2 to 25, 2 to 30 nucleotides of the oligonucleotide are nucleotide analogues.

The oligonucleotide may be of 8 to 15 nucleotides in length in which 2 to 4, 2 to 5, 2 to 6, 2 to 7, 2 to 8, 2 to 9, 2 to 10, 2 to 11, 2 to 12, 2 to 13, 2 to 14 nucleotides of the oligonucleotide are nucleotide analogues. Optionally, the oligonucleotides may have every nucleotide except 1, 2, 3, 4, 5, 6, 7, 8, 9, or 10 nucleotides modified.

The oligonucleotide may consist entirely of bridged nucleotides (e.g., LNA nucleotides, cEt nucleotides, ENA nucleotides). The oligonucleotide may comprise alternating deoxyribonucleotides and 2′-fluoro-deoxyribonucleotides. The oligonucleotide may comprise alternating deoxyribonucleotides and 2′-O-methyl nucleotides. The oligonucleotide may comprise alternating deoxyribonucleotides and ENA nucleotide analogues. The oligonucleotide may comprise alternating deoxyribonucleotides and LNA nucleotides. The oligonucleotide may comprise alternating LNA nucleotides and 2′-O-methyl nucleotides. The oligonucleotide may have a 5′ nucleotide that is a bridged nucleotide (e.g., a LNA nucleotide, cEt nucleotide, ENA nucleotide). The oligonucleotide may have a 5′ nucleotide that is a deoxyribonucleotide.

The oligonucleotide may comprise deoxyribonucleotides flanked by at least one bridged nucleotide (e.g., a LNA nucleotide, cEt nucleotide, ENA nucleotide) on each of the 5′ and 3′ ends of the deoxyribonucleotides. The oligonucleotide may comprise deoxyribonucleotides flanked by 1, 2, 3, 4, 5, 6, 7, 8 or more bridged nucleotides (e.g., LNA nucleotides, cEt nucleotides, ENA nucleotides) on each of the 5′ and 3′ ends of the deoxyribonucleotides. The 3′ position of the oligonucleotide may have a 3′ hydroxyl group. The 3′ position of the oligonucleotide may have a 3′ thiophosphate.

The oligonucleotide may be conjugated with a label. For example, the oligonucleotide may be conjugated with a biotin moiety, cholesterol, Vitamin A, folate, sigma receptor ligands, aptamers, peptides, such as CPP, hydrophobic molecules, such as lipids, ligands of the asialoglycoprotein receptor (ASGPR), such as GalNac, or dynamic polyconjugates and variants thereof at its 5′ or 3′ end.

Preferably an oligonucleotide comprises one or more modifications comprising: a modified sugar moiety, and/or a modified internucleoside linkage, and/or a modified nucleotide and/or combinations thereof. It is not necessary for all positions in a given oligonucleotide to be uniformly modified, and in fact more than one of the modifications described herein may be incorporated in a single oligonucleotide or even at within a single nucleoside within an oligonucleotide.

In some embodiments, the oligonucleotides are chimeric oligonucleotides that contain two or more chemically distinct regions, each made up of at least one nucleotide. These oligonucleotides typically contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids. Chimeric oligonucleotides of the disclosure may be formed as composite structures of two or more oligonucleotides, modified oligonucleotides, oligonucleosides and/or oligonucleotide mimetics as described above. Such compounds have also been referred to in the art as hybrids or gapmers. Representative United States patents that teach the preparation of such hybrid structures comprise, but are not limited to, U.S. Pat. Nos. 5,013,830; 5,149,797; 5, 220,007; 5,256,775; 5,366,878; 5,403,711; 5,491,133; 5,565,350; 5,623,065; 5,652,355; 5,652,356; and 5,700,922, each of which is herein incorporated by reference.

In some embodiments, an oligonucleotide comprises at least one nucleotide modified at the 2′ position of the sugar, most preferably a 2′-O-alkyl, 2′-O-alkyl-O-alkyl or 2′-fluoro-modified nucleotide. In other preferred embodiments, RNA modifications include 2′-fluoro, 2′-amino and 2′ O-methyl modifications on the ribose of pyrimidines, abasic residues or an inverted base at the 3′ end of the RNA. Such modifications are routinely incorporated into oligonucleotides and these oligonucleotides have been shown to have a higher Tm (e.g., higher target binding affinity) than 2′-deoxyoligonucleotides against a given target.

A number of nucleotide and nucleoside modifications have been shown to make the oligonucleotide into which they are incorporated more resistant to nuclease digestion than the native oligodeoxynucleotide; these modified oligos survive intact for a longer time than unmodified oligonucleotides. Specific examples of modified oligonucleotides include those comprising modified backbones, for example, phosphorothioates, phosphotriesters, methyl phosphonates, short chain alkyl or cycloalkyl intersugar linkages or short chain heteroatomic or heterocyclic intersugar linkages. In some embodiments, oligonucleotides may have phosphorothioate backbones; heteroatom backbones, such as methylene(methylimino) or MMI backbones; amide backbones (see De Mesmaeker et al. Ace. Chem. Res. 1995, 28:366-374); morpholino backbones (see Summerton and Weller, U.S. Pat. No. 5,034,506); or peptide nucleic acid (PNA) backbones (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050.

Morpholino-based oligomeric compounds are described in Dwaine A. Braasch and David R. Corey, Biochemistry, 2002, 41(14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991. In some embodiments, the morpholino-based oligomeric compound is a phosphorodiamidate morpholino oligomer (PMO) (e.g., as described in Iverson, Curr. Opin. Mol. Ther., 3:235-238, 2001; and Wang et al., J. Gene Med., 12:354-364, 2010; the disclosures of which are incorporated herein by reference in their entireties).

Cyclohexenyl nucleic acid oligonucleotide mimetics are described in Wang et al., J. Am. Chem. Soc., 2000, 122, 8595-8602.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts; see U.S. Pat. Nos. 5,034,506; 5,166,315; 5,185,444; 5,214,134; 5,216,141; 5,235,033; 5,264, 562; 5, 264,564; 5,405,938; 5,434,257; 5,466,677; 5,470,967; 5,489,677; 5,541,307; 5,561,225; 5,596, 086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

Modified oligonucleotides are also known that include oligonucleotides that are based on or constructed from arabinonucleotide or modified arabinonucleotide residues. Arabinonucleosides are stereoisomers of ribonucleosides, differing only in the configuration at the 2′-position of the sugar ring. In some embodiments, a 2′-arabino modification is 2′-F arabino. In some embodiments, the modified oligonucleotide is 2′-fluoro-D-arabinonucleic acid (FANA) (as described in, for example, Lon et al., Biochem., 41:3457-3467, 2002 and Min et al., Bioorg. Med. Chem. Lett., 12:2651-2654, 2002; the disclosures of which are incorporated herein by reference in their entireties). Similar modifications can also be made at other positions on the sugar, particularly the 3′ position of the sugar on a 3′ terminal nucleoside or in 2′-5′ linked oligonucleotides and the 5′ position of 5′ terminal nucleotide.

PCT Publication No. WO 99/67378 discloses arabinonucleic acids (ANA) oligomers and their analogues for improved sequence specific inhibition of gene expression via association to complementary messenger RNA.

Other modifications include ethylene-bridged nucleic acids (ENAs) (e.g., International Patent Publication No. WO 2005/042777, Morita et al., Nucleic Acid Res., Suppl 1:241-242, 2001; Surono et al., Hum. Gene Ther., 15:749-757, 2004; Koizumi, Curr. Opin. Mol. Ther., 8:144-149, 2006 and Horie et al., Nucleic Acids Symp. Ser (Oxf), 49:171-172, 2005; the disclosures of which are incorporated herein by reference in their entireties). Preferred ENAs include, but are not limited to, 2′-O,4′-C-ethylene-bridged nucleic acids.

Examples of LNAs are described in WO/2008/043753.

In some embodiments, the Locked Nucleic Acid (LNA) used in the oligonucleotides described herein comprises at least one Locked Nucleic Acid (LNA) unit according any of the formulas shown in Scheme 2 of PCT/DK2006/000512.

In some embodiments, the LNA used in the oligomer of the disclosure comprises internucleoside linkages selected from -0-P(O)₂—O—, —O—P(O,S)—O—, -0-P(S)₂—O—, —S—P(O)₂—O—, —S—P(O,S)—O—, —S—P(S)₂—O—, -0-P(O)₂—S—, —O—P(O,S)—S—, —S—P(O)₂—S—, —O—PO(R^{H})—O—, O—PO(OCH₃)—O—, —O—PO(NR^{H})—O—, —O—PO(OCH₂CH₂S—R)—O—, —O—PO(BH₃)—O—, PO(NHR^{H})—O—, —O—P(O)₂—NR^{H}—, —NR^{H}—P(O)₂—O—, —NR^{H}—CO—O—, where R^{H} is selected from hydrogen and C₁₋₄-alkyl. Oligonucleotides can also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, isocytosine, pseudoisocytosine, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 5-propynyluracil, 8-azaguanine, 7-deazaguanine, N6 (6-aminohexyl)adenine, 6-aminopurine, 2-aminopurine, 2-chloro-6-aminopurine and 2,6-diaminopurine or other diaminopurines. See, e.g., Kornberg, “DNA Replication,” W. H. Freeman & Co., San Francisco, 1980, pp 75-′7′7; and Gebeyehu, G., et al. Nucl. Acids Res., 15:4513 (1987)). A “universal” base known in the art, e.g., inosine, can also be included. 5-Me-C substitutions have been shown to increase nucleic acid duplex stability by 0.6-1.2° C. (Sanghvi, in Crooke, and Lebleu, eds., Antisense Research and Applications, CRC Press, Boca Raton, 1993, pp. 276-278) and may be used as base substitutions.

It is not necessary for all positions in a given oligonucleotide to be uniformly modified, and in fact more than one of the modifications described herein may be incorporated in a single oligonucleotide or even at within a single nucleoside within an oligonucleotide.

In some embodiments, both a sugar and an internucleoside linkage, e.g., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al, Science, 1991, 254, 1497-1500.

Oligonucleotides can also include one or more nucleobase (often referred to in the art simply as “base”) modifications or substitutions. As used herein, “unmodified” or “natural” nucleobases comprise the purine bases adenine (A) and guanine (G), and the pyrimidine bases thymine (T), cytosine (C) and uracil (U). Modified nucleobases comprise other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine and 3-deazaguanine and 3-deazaadenine.

Further, nucleobases comprise those disclosed in U.S. Pat. No. 3,687,808, those disclosed in “The Concise Encyclopedia of Polymer Science And Engineering”, pages 858-859, Kroschwitz, ed. John Wiley & Sons, 1990; those disclosed by Englisch et al., Angewandle Chemie, International Edition, 1991, 30, page 613, and those disclosed by Sanghvi, Chapter 15, Antisense Research and Applications,” pages 289-302, Crooke, and Lebleu, eds., CRC Press, 1993. Certain of these nucleobases are particularly useful for increasing the binding affinity of the oligomeric compounds of the disclosure. These include 5-substituted pyrimidines, 6-azapyrimidines and N-2, N-6 and 0-6 substituted purines, comprising 2-aminopropyladenine, 5-propynyluracil and 5-propynylcytosine. 5-methylcytosine substitutions have been shown to increase nucleic acid duplex stability by 0.6-1.2<0>C (Sanghvi, et al., eds, “Antisense Research and Applications,” CRC Press, Boca Raton, 1993, pp. 276-278) and are presently preferred base substitutions, even more particularly when combined with 2′-O-methoxyethyl sugar modifications. Modified nucleobases are described in U.S. Pat. No. 3,687,808, as well as U.S. Pat. Nos. 4,845,205; 5,130,302; 5,134,066; 5,175, 273; 5, 367,066; 5,432,272; 5,457,187; 5,459,255; 5,484,908; 5,502,177; 5,525,711; 5,552,540; 5,587,469; 5,596,091; 5,614,617; 5,750,692, and 5,681,941, each of which is herein incorporated by reference.

In some embodiments, the oligonucleotides are chemically linked to one or more moieties or conjugates that enhance the activity, cellular distribution, or cellular uptake of the oligonucleotide. For example, one or more oligonucleotides, of the same or different types, can be conjugated to each other; or oligonucleotides can be conjugated to targeting moieties with enhanced specificity for a cell type or tissue type. Such moieties include, but are not limited to, lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al, Ann. N. Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Mancharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-t oxycholesterol moiety (Crooke et al., J. Pharmacol. Exp. Ther., 1996, 277, 923-937). In some embodiments, oligonucleotide modification include modification of the 5′ or 3′ end of the oligonucleotide. In some embodiments, the 3′ end of the oligonucleotide comprises a hydroxyl group or a thiophosphate. It should be appreciated that additional molecules (e.g. a biotin moiety or a fluorophor) can be conjugated to the 5′ or 3′ end of an oligonucleotide. In some embodiments, an oligonucleotide comprises a biotin moiety conjugated to the 5′ nucleotide.

In some embodiments, an oligonucleotide comprises locked nucleic acids (LNA), ENA modified nucleotides, 2′-O-methyl nucleotides, or 2′-fluoro-deoxyribonucleotides. In some embodiments, an oligonucleotide comprises alternating deoxyribonucleotides and 2′-fluoro-deoxyribonucleotides. In some embodiments, an oligonucleotide comprises alternating deoxyribonucleotides and 2′-O-methyl nucleotides. In some embodiments, an oligonucleotide comprises alternating deoxyribonucleotides and ENA modified nucleotides. In some embodiments, an oligonucleotide comprises alternating deoxyribonucleotides and locked nucleic acid nucleotides. In some embodiments, an oligonucleotide comprises alternating locked nucleic acid nucleotides and 2′-O-methyl nucleotides.

In some embodiments, the 5′ nucleotide of the oligonucleotide is a deoxyribonucleotide. In some embodiments, the 5′ nucleotide of the oligonucleotide is a locked nucleic acid nucleotide. In some embodiments, the nucleotides of the oligonucleotide comprise deoxyribonucleotides flanked by at least one locked nucleic acid nucleotide on each of the 5′ and 3′ ends of the deoxyribonucleotides. In some embodiments, the nucleotide at the 3′ position of the oligonucleotide has a 3′ hydroxyl group or a 3′ thiophosphate.

In some embodiments, an oligonucleotide comprises phosphorothioate internucleotide linkages. In some embodiments, an oligonucleotide comprises phosphorothioate internucleotide linkages between at least two nucleotides. In some embodiments, an oligonucleotide comprises phosphorothioate internucleotide linkages between all nucleotides.

It should be appreciated that an oligonucleotide can have any combination of modifications as described herein.

### IV. FORMULATIONS

a. Lipid Nanoparticles

A suitable lipid nanoparticle for the present disclosure may include on or more of any of the cationic lipids, non-cationic lipids, and/or PEG-modified lipids described herein at various ratios. Typically, a lipid nanoparticle in accordance with the present disclosure comprises a cationic lipid, a non-cationic lipid, and a PEG-modified lipid. The formulations described herein include a multi-component lipid mixture of varying ratios employing one or more cationic lipids, non-cationic lipids and PEG-modified lipids designed to encapsulate an oligonucleotide that is complementary to a target sequence.

In some embodiments, the ratio of cationic lipid(s) to non-cationic lipid(s) to PEG-modified lipids may be between about 30-60:30-45:1-15, respectively. In some embodiments, the ratio of cationic lipid(s) to non-cationic lipid(s) to PEG-modified lipids is approximately 60:45:5, respectively. In some embodiments, the ratio of cationic lipid(s) to non-cationic lipid(s) to PEG-modified lipid(s) is approximately 50:35:15, respectively. In some embodiments, the ratio of cationic lipid(s) to non-cationic lipid(s) to PEG-modified lipid(s) is approximately 40:45:15, respectively.

The diameter of a lipid nanoparticle of the present disclosure is measured by dynamic light scattering (DLS). This technique is well-known in the art, see, e.g., Ramos, 2017, Dynamic Light Scattering Applied to Nanoparticle Characterization, Micro and Nano Technologies, 99-110; Horiba, 2018, Dynamic Light Scattering Technology; Carvalho, et al., 2018, Application of Light Scattering Techniques to Nanoparticle Characterization and Development, Front. Chem. Briefly, the lipid nanoparticle size is determined by measuring the random changes in the intensity of laser light that is scattering from the formulation.

In some embodiments, the lipid nanoparticles of the present disclosure have a mean diameter of 50 nm to 150 nm, e.g., 60 nm to 130 nm, 70 nm to 110 nm, 60 nm to 80 nm, e.g., as measured by dynamic light scattering. In some embodiments, the lipid nanoparticles of the present disclosure have a mean diameter of about 20-50 nm, e.g., as measured by dynamic light scattering. In some embodiments, the lipid nanoparticles of the present disclosure have a mean diameter of about 30 nm. In some embodiments, the particle size is at least 30 nm, at least 40 nm, at least 50 nm, at least 60 nm, at least 70 nm, at least 80 nm, at least 90 nm, at least 100 nm, at least 110 nm, or at least 120 nm, e.g., as measured by dynamic light scattering. In some embodiments, the particle size is less than 30 nm, less than 40 nm, less than 50 nm, less than 60 nm, less than 70 nm, less than 80 nm, less than 90 nm, less than 100 nm, less than 110 nm, or less than 120 nm, e.g., as measured by dynamic light scattering.

In some embodiments, the oligonucleotide is encapsulated in the lipid nanoparticle. In some embodiments, the oligonucleotide is associated on both the surface of the lipid nanoparticle and encapsulated within the same lipid nanoparticle. For example, during preparation of the oligonucleotide encapsulated in a lipid nanoparticle, cationic lipids may associate with the oligonucleotides through electrostatic interactions. For example, during preparation of the lipid nanoparticles of the disclosure, cationic lipids may associate with the oligonucleotide through electrostatic interactions.

In some embodiments, the one or more oligonucleotide species may be encapsulated in the same lipid nanoparticle. In some embodiments, the one or more oligonucleotide species may be encapsulated in different lipid nanoparticles. In some embodiments, the oligonucleotide is encapsulated in one or more lipid nanoparticles, which differ in their lipid composition, molar ratio of lipid components, size, charge (Zeta potential), targeting ligands and/or combinations thereof. In some embodiments, the one or more lipid nanoparticles may have a different composition of cationic lipids, non-cationic lipid, PEG-modified lipid and/or combinations thereof. In some embodiments the one or more lipid nanoparticles may have a different molar ratio of cationic lipid, non-cationic lipid, and PEG-modified lipid used to create the lipid nanoparticle.

In some embodiments, oligonucleotides are encapsulated in the lipid nanoparticle of the present disclosure. In some embodiments, encapsulated oligonucleotides are resistant in aqueous solution to degradation with a nuclease. In some embodiments, the oligonucleotides encapsulated in the particles are not significantly degraded after exposure to serum or a nuclease assay that would significantly degrade free nucleic acid. Nuclease degradation of nucleic acids may be determined by an Oligreen® assay (Invitrogen Corporation, Carlsbad, Calif.), which is an ultra-sensitive fluorescent nucleic acid stain for quantitating oligonucleotides and single-stranded DNA in solution. In some embodiments, oligonucleotides are at least 50%, at least 60%, at least 70%, at least 80%, at least 90%, at least 95%, or at least 97% encapsulated in the lipid nanoparticle.

In one embodiment, the lipid to drug ratio (mass/mass ratio; w/w ratio) (e.g., lipid to dsRNA ratio) will be in the range of from about 1:1 to about 50:1, from about 1:1 to about 25:1, from about 10:1 to about 14:1, from about 3:1 to about 15:1, from about 4:1 to about 10:1, from about 5:1 to about 9:1, or about 6:1 to about 9:1.

In some embodiments, a particle of the present disclosure comprises a polymer. In some embodiments, the polymer comprises a layer of a hydrogel or surgical sealant. In some embodiments, the polymer is PLGA, ethylene vinyl acetate, poloxamer, GELSITE®, or a combination thereof. In some embodiments, a particle of the present disclosure comprises a fibrin sealant.

A formulated oligonucleotide and lipid nanoparticle composition can assume a variety of states. In some examples, the composition is at least partially crystalline, uniformly crystalline, and/or anhydrous (e.g., less than 80, 50, 30, 20, or 10% water). In another example, an oligonucleotide is in an aqueous phase, e.g., in a solution that includes water. The aqueous phase or the crystalline compositions can, e.g., be incorporated into a delivery vehicle, e.g., a lipid nanoparticle (particularly for the aqueous phase) or a particle (e.g., a microparticle as can be appropriate for a crystalline composition) as described herein. Generally, an oligonucleotide composition is formulated in a manner that is compatible with the intended method of administration.

An oligonucleotide preparation can be formulated or administered (together or separately) in combination with another agent, e.g., another therapeutic agent or an agent that stabilizes an oligonucleotide, e.g., a protein that complexes with oligonucleotide. Still other agents include chelators, e.g., EDTA (e.g., to remove divalent cations such as Mg²⁺), salts, RNAse inhibitors (e.g., a broad specificity RNAse inhibitor such as RNAsin) and so forth.

b. Manufacture

The lipid nanoparticles used in the methods of the present disclosure can be prepared by various techniques which are presently known in the art. For example, multilamellar vesicles (MLV) may be prepared according to conventional techniques, such as by depositing a selected lipid on the inside wall of a suitable container or vessel by dissolving the lipid in an appropriate solvent, and then evaporating the solvent to leave a thin film on the inside of the vessel or by spray drying. An aqueous phase then may be added to the vessel with a vortexing motion which results in the formation of MLVs. Unilamellar vesicles (ULV) can then be formed by homogenization, sonication or extrusion of the multilamellar vesicles. In addition, unilamellar vesicles can be formed by detergent removal techniques.

In some embodiments, the lipid nanoparticles is prepared by at least one of the following methods: aerosolization, spray drying, lyophilization, vacuum drying, evaporation, fluid bed drying, or a combination of these techniques; or sonication with a lipid, freeze-drying, condensation and other self-assembly.

The process of incorporation of a desired oligonucleotide into a lipid nanoparticle is often referred to as “loading”. Exemplary methods are described in Lasic, et al., FEBS Lett., 312: 255-258, 1992, which is incorporated herein by reference. In a typical embodiment, the oligonucleotide of the disclosure is encapsulated in a lipid nanoparticle using the methods described in WO 2018/089801 (the teachings of which are incorporated herein by reference in their entirety). Briefly, the oligonucleotide is encapsulated by mixing of a solution comprising pre-formed lipid nanoparticles with oligonucleotide such that lipid nanoparticles encapsulating oligonucleotide are formed.

In some embodiments, it is desirable to precondense the oligonucleotide into the core of a lipid nanoparticle described herein. In some embodiments, a helper cationic polymer, such as protamine is included in the formulation. In some embodiments, protamine interacts with nucleic acid to form a negatively charged compact core.

Typically, the lipid nanoparticles-incorporated oligonucleotide is completely located in the interior space of the lipid nanoparticle, although as discussed above, some of the oligonucleotide (e.g., no more than 10% of total oligonucleotide in the nanoparticle composition) may also be associated with the exterior surface of the lipid nanoparticle membrane. The incorporation of an oligonucleotide into lipid nanoparticles is also referred to herein as “encapsulation”. Typically, the purpose of incorporating an oligonucleotide into a nanoparticle is to protect the nucleic acid from an environment which may contain enzymes or chemicals that degrade nucleic acids and/or systems or receptors that cause the rapid excretion of the oligonucleotide. Accordingly, in some embodiments, a suitable delivery vehicle is capable of enhancing the stability of the oligonucleotide contained therein and/or facilitate the delivery of oligonucleotide to the target cell or tissue.

c. Cationic Lipids

Cationic lipids are any of a number of lipid and lipidoid species that have a net positive charge at a selected pH, such as at physiological pH. Several cationic lipids have been described in the literature, many of which are commercially available.

Suitable cationic lipids for use in the formulations and methods provided herein include the cationic lipids as described in International Patent Publication WO 2010/144740, which is incorporated herein by reference. In certain embodiments, the compositions and methods of the present invention include a cationic lipid, (6Z,9Z,28Z,31Z)-heptatriaconta-6,9,28,31-tetraen-19-yl 4-(dimethylamino) butanoate, having a compound structure of:

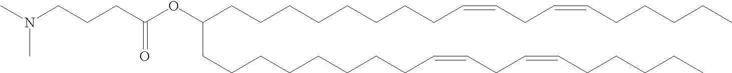 [SMILES: CCCCC/C=C\C/C=C\CCCCCCCCC(CCCCCCCC/C=C\C/C=C\CCCCC)OC(=O)CCCN(C)C]

and pharmaceutically acceptable salts thereof.

Other suitable cationic lipids for use in the compositions and methods of the present disclosure include ionizable cationic lipids as described in International Patent Publication WO 2013/149140, which is incorporated herein by reference. In some embodiments, the compositions and methods of the present disclosure include a cationic lipid of one of the following formulas:

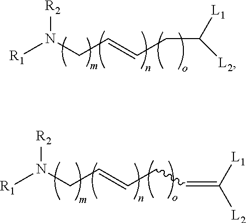

or a pharmaceutically acceptable salt thereof, wherein R₁ and R₂ are each independently selected from the group consisting of hydrogen, an optionally substituted, variably saturated or unsaturated C₁-C₂₀ alkyl and an optionally substituted, variably saturated or unsaturated C₆-C₂₀ acyl; wherein L₁ and L₂ are each independently selected from the group consisting of hydrogen, an optionally substituted C₁-C₃₀ alkyl, an optionally substituted variably unsaturated C₁-C₃₀ alkenyl, and an optionally substituted C₁-C₃₀ alkynyl; wherein m and o are each independently selected from the group consisting of zero and any positive integer (e.g., where m is three); and wherein n is zero or any positive integer (e.g., where n is one). In certain embodiments, the compositions and methods of the present disclosure include the cationic lipid (15Z,18Z)—N,N-dimethyl-6-(9Z,12Z)-octadeca-9,12-dien-1-yl) tetracosa-15,18-dien-1-amine (“HGT5000”), having a compound structure of:

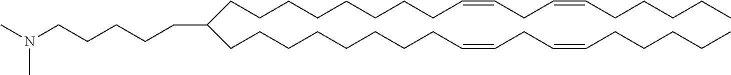 [SMILES: CCCCC/C=C\C/C=C\CCCCCCCCC(CCCCCCCC/C=C\C/C=C\CCCCC)CCCCCN(C)C]

### (HGT-5000)

and pharmaceutically acceptable salts thereof. In certain embodiments, the compositions and methods of the present disclosure include the cationic lipid (15Z,18Z)—N,N-dimethyl-6-((9Z,12Z)-octadeca-9,12-dien-1-yl) tetracosa-4,15,18-trien-1-amine (“HGT5001”), having a compound structure of:

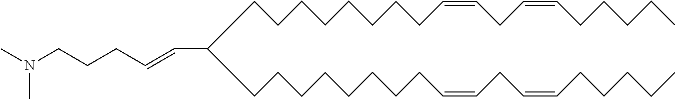 [SMILES: CCCCC/C=C\C/C=C\CCCCCCCCCC(/C=C/CCCN(C)C)CCCCCCCCC/C=C\C/C=C\CCCCC]

### (HGT-5001)

and pharmaceutically acceptable salts thereof. In certain embodiments, the compositions and methods of the present disclosure include the cationic lipid and (15Z,18Z)—N,N-dimethyl-6-((9Z,12Z)-octadeca-9,12-dien-1-yl) tetracosa-5,15,18-trien-1-amine (“HGT5002”), having a compound structure of:

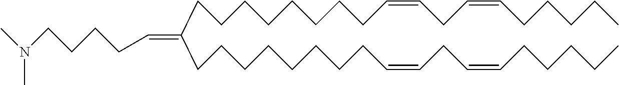 [SMILES: CCCCC/C=C\C/C=C\CCCCCCCCC(=CCCCCN(C)C)CCCCCCCC/C=C\C/C=C\CCCCC]

(HGT-5002) and pharmaceutically acceptable salts thereof.

Other suitable cationic lipids for use in the compositions and methods of the disclosure include cationic lipids described as aminoalcohol lipidoids in International Patent Publication WO 2010/053572, which is incorporated herein by reference. In certain embodiments, the compositions and methods of the present disclosure include a cationic lipid having a compound structure of:

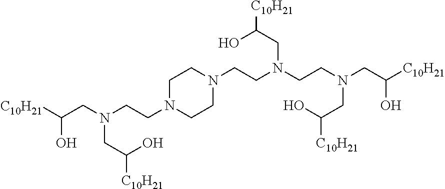

and pharmaceutically acceptable salts thereof.

Other suitable cationic lipids for use in the compositions and methods of the disclosure include the cationic lipids as described in International Patent Publication WO 2016/118725, which is incorporated herein by reference. In certain embodiments, the compositions and methods of the present disclosure include a cationic lipid having a compound structure of:

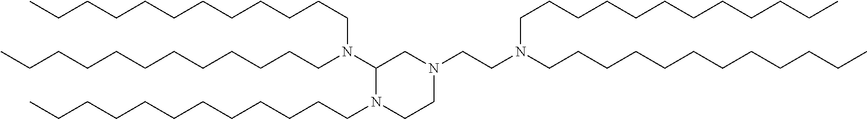 [SMILES: CCCCCCCCCCCCN(CCCCCCCCCCCC)CCN1CCN(CCCCCCCCCCCC)C(N(CCCCCCCCCCCC)CCCCCCCCCCCC)C1]

and pharmaceutically acceptable salts thereof.

Other suitable cationic lipids for use in the compositions and methods of the disclosure include the cationic lipids as described in International Patent Publication WO 2016/118724, which is incorporated herein by reference. In certain embodiments, the compositions and methods of the present disclosure include a cationic lipid having a compound structure of:

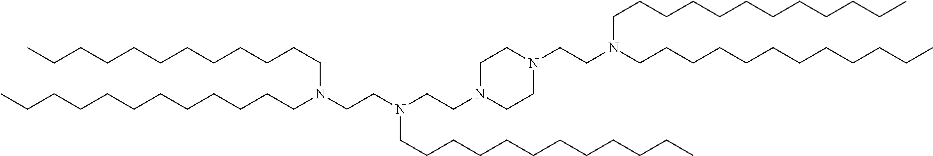 [SMILES: CCCCCCCCCCCCN(CCCCCCCCCCCC)CCN(CCCCCCCCCCCC)CCN1CCN(CCN(CCCCCCCCCCCC)CCCCCCCCCCCC)CC1]

and pharmaceutically acceptable salts thereof.

Other suitable cationic lipids for use in the compositions and methods of the disclosure include a cationic lipid having the formula of 14,25-ditridecyl 15,18,21,24-tetraaza-octatriacontane, and pharmaceutically acceptable salts thereof.

Other suitable cationic lipids for use in the compositions and methods of the disclosure include the cationic lipids as described in International Patent Publications WO 2013/063468 and WO 2016/205691, each of which are incorporated herein by reference. In some embodiments, the compositions and methods of the present disclosure include a cationic lipid of the following formula:

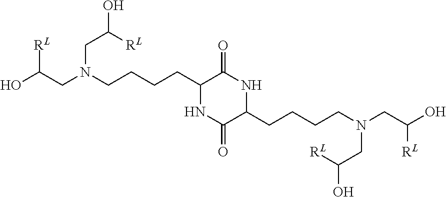

or pharmaceutically acceptable salts thereof, wherein each instance of R^{L} is independently optionally substituted C₆-C₄₀ alkenyl. In certain embodiments, the compositions and methods of the present disclosure include a cationic lipid having a compound structure of:

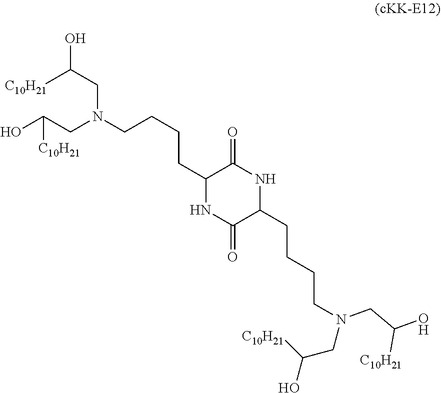

and pharmaceutically acceptable salts thereof. In certain embodiments, the compositions and methods of the present disclosure include a cationic lipid having a compound structure of:

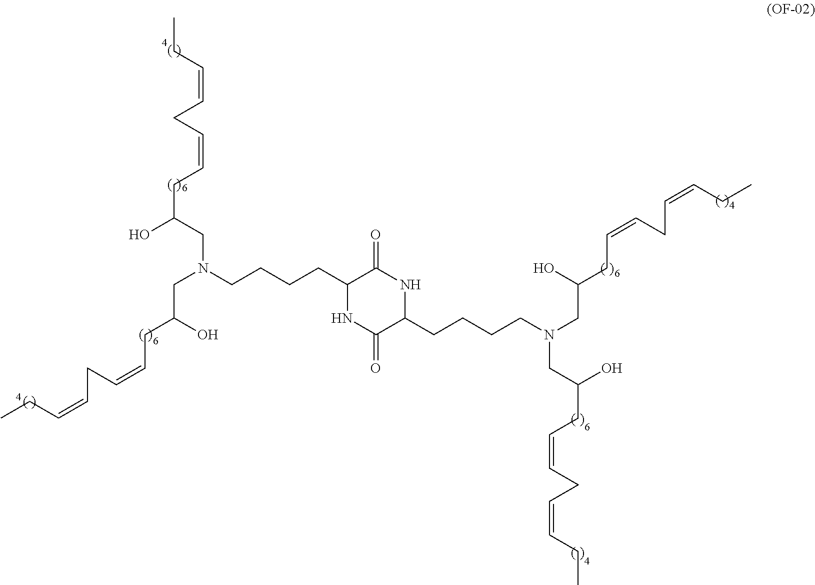 [SMILES: CC/C=C\C/C=C\CC(O)CN(CCCCC1NC(=O)C(CCCCN(CC(O)C/C=C\C/C=C\CC)CC(O)C/C=C\C/C=C\CC)NC1=O)CC(O)C/C=C\C/C=C\CC]

and pharmaceutically acceptable salts thereof. In certain embodiments, the compositions and methods of the present disclosure include a cationic lipid having a compound structure of:

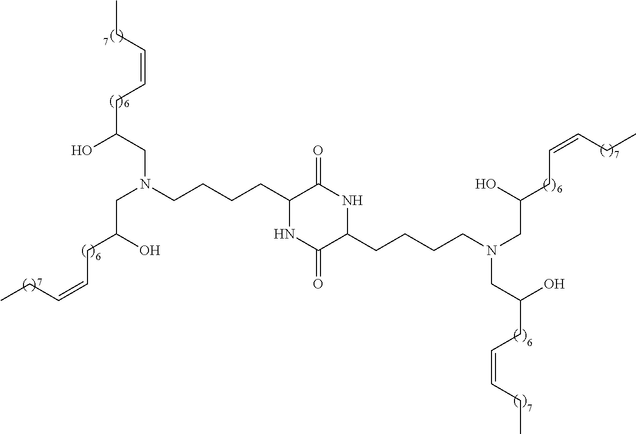 [SMILES: CC/C=C\CC(O)CN(CCCCC1NC(=O)C(CCCCN(CC(O)C/C=C\CC)CC(O)C/C=C\CC)NC1=O)CC(O)C/C=C\CC]

and pharmaceutically acceptable salts thereof. In certain embodiments, the compositions and methods of the present disclosure include a cationic lipid having a compound structure of:

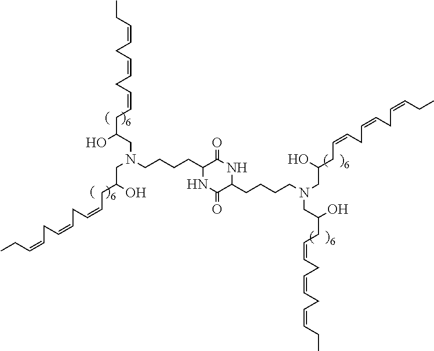

and pharmaceutically acceptable salts thereof.

Other suitable cationic lipids for use in the compositions and methods of the disclosure include the cationic lipids as described in International Patent Publication WO 2015/184256, which is incorporated herein by reference. In some embodiments, the compositions and methods of the present disclosure include a cationic lipid of the following formula:

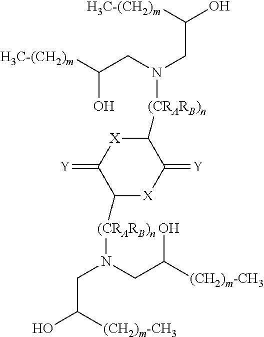

or a pharmaceutically acceptable salt thereof, wherein each X independently is O or S; each Y independently is O or S; each m independently is 0 to 20; each n independently is 1 to 6; each R_{A} is independently hydrogen, optionally substituted C1-50 alkyl, optionally substituted C2-50 alkenyl, optionally substituted C2-50 alkynyl, optionally substituted C3-10 carbocyclyl, optionally substituted 3-14 membered heterocyclyl, optionally substituted C6-14 aryl, optionally substituted 5-14 membered heteroaryl or halogen; and each RB is independently hydrogen, optionally substituted C1-50 alkyl, optionally substituted C2-50 alkenyl, optionally substituted C2-50 alkynyl, optionally substituted C3-10 carbocyclyl, optionally substituted 3-14 membered heterocyclyl, optionally substituted C6-14 aryl, optionally substituted 5-14 membered heteroaryl or halogen. In certain embodiments, the compositions and methods of the present disclosure include a cationic lipid, “Target 23”, having a compound structure of:

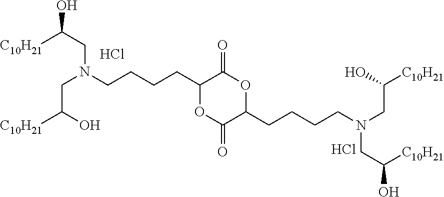

### (Target 23)

and pharmaceutically acceptable salts thereof.

Other suitable cationic lipids for use in the compositions and methods of the disclosure include the cationic lipids as described in International Patent Publication WO 2016/004202, which is incorporated herein by reference. In some embodiments, the compositions and methods of the present disclosure include a cationic lipid having the compound structure:

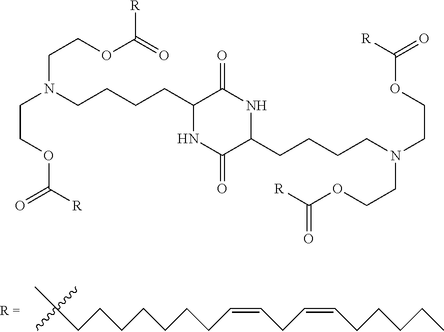

or a pharmaceutically acceptable salt thereof. In some embodiments, the compositions and methods of the present disclosure include a cationic lipid having the compound structure:

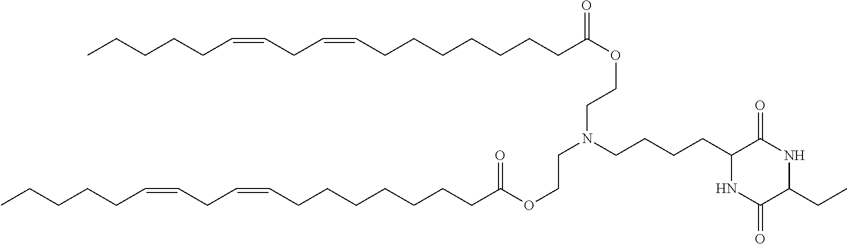 [SMILES: CCCCC/C=C\C/C=C\CCCCCCCC(=O)OCCN(CCCCC1NC(=O)C(CC)NC1=O)CCOC(=O)CCCCCCC/C=C\C/C=C\CCCCC]

or a pharmaceutically acceptable salt thereof. In some embodiments, the compositions and methods of the present disclosure include a cationic lipid having the compound structure:

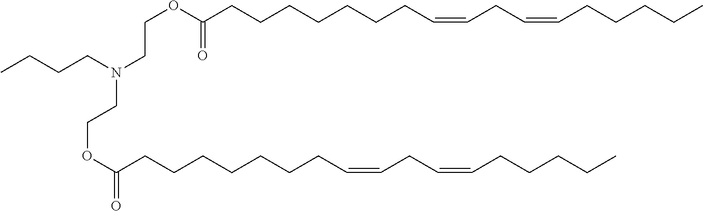 [SMILES: CCCCC/C=C\C/C=C\CCCCCCCC(=O)OCCN(CCCC)CCOC(=O)CCCCCCC/C=C\C/C=C\CCCCC]

or a pharmaceutically acceptable salt thereof.

Other suitable cationic lipids for use in the compositions and methods of the present disclosure include the cationic lipids as described in J. McClellan, M. C. King, Cell 2010, 141, 210-217 and in Whitehead et al., Nature Communications (2014) 5:4277, which is incorporated herein by reference. In certain embodiments, the cationic lipids of the compositions and methods of the present disclosure include a cationic lipid having a compound structure of:

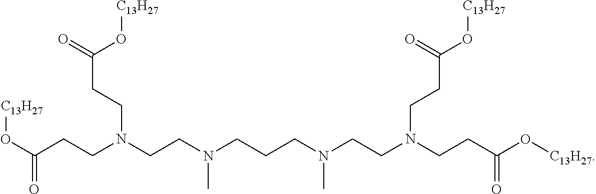

and pharmaceutically acceptable salts thereof.

Other suitable cationic lipids for use in the compositions and methods of the disclosure include the cationic lipids as described in International Patent Publication WO 2015/199952, which is incorporated herein by reference. In some embodiments, the compositions and methods of the present disclosure include a cationic lipid having the compound structure:

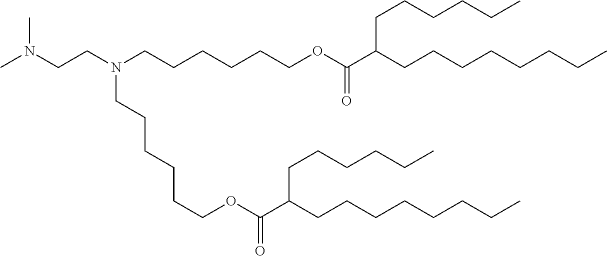 [SMILES: CCCCCCCCC(CCCCCC)C(=O)OCCCCCCN(CCCCCCOC(=O)C(CCCCCC)CCCCCCCC)CCN(C)C]

and pharmaceutically acceptable salts thereof. In some embodiments, the compositions and methods of the present disclosure include a cationic lipid having the compound structure:

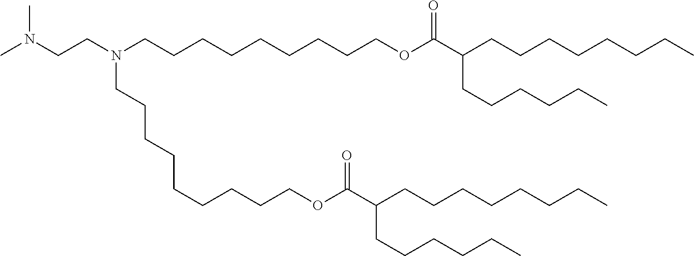 [SMILES: CCCCCCCCC(CCCCCC)C(=O)OCCCCCCCCCN(CCCCCCCCCOC(=O)C(CCCCCC)CCCCCCCC)CCN(C)C]

and pharmaceutically acceptable salts thereof. In some embodiments, the compositions and methods of the present disclosure include a cationic lipid having the compound structure:

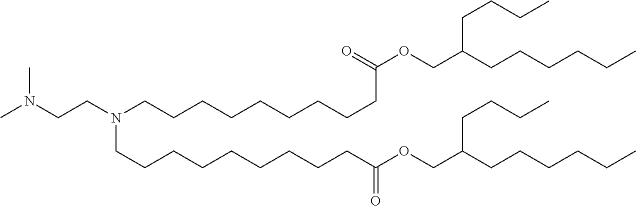 [SMILES: CCCCCCC(CCCC)COC(=O)CCCCCCCCCN(CCCCCCCCCC(=O)OCC(CCCC)CCCCCC)CCN(C)C]

and pharmaceutically acceptable salts thereof. In some embodiments, the compositions and methods of the present disclosure include a cationic lipid having the compound structure:

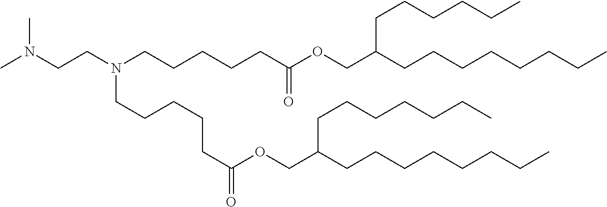 [SMILES: CCCCCCCCC(CCCCCC)COC(=O)CCCCCN(CCCCCC(=O)OCC(CCCCCCC)CCCCCCCC)CCN(C)C]

and pharmaceutically acceptable salts thereof. In some embodiments, the compositions and methods of the present disclosure include a cationic lipid having the compound structure:

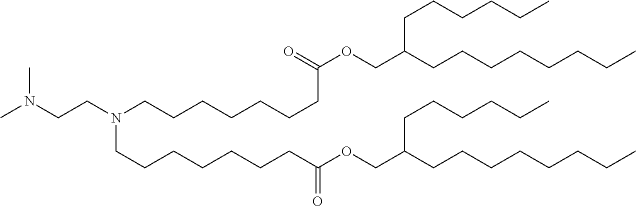 [SMILES: CCCCCCCCC(CCCCCC)COC(=O)CCCCCCCN(CCCCCCCC(=O)OCC(CCCCCC)CCCCCCCC)CCN(C)C]

and pharmaceutically acceptable salts thereof. In some embodiments, the compositions and methods of the present disclosure include a cationic lipid having the compound structure:

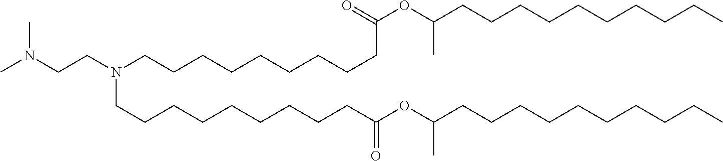 [SMILES: CCCCCCCCCCC(C)OC(=O)CCCCCCCCCN(CCCCCCCCCC(=O)OC(C)CCCCCCCCCC)CCN(C)C]

and pharmaceutically acceptable salts thereof. In some embodiments, the compositions and methods of the present disclosure include a cationic lipid having the compound structure:

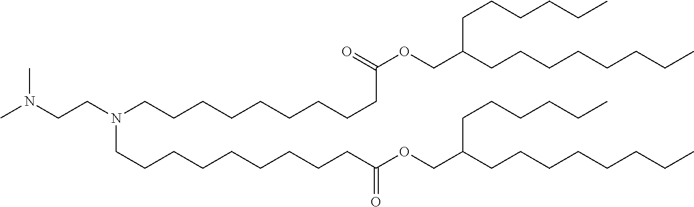 [SMILES: CCCCCCCCC(CCCCCC)COC(=O)CCCCCCCCCN(CCCCCCCCCC(=O)OCC(CCCCCC)CCCCCCCC)CCN(C)C]

and pharmaceutically acceptable salts thereof. In some embodiments, the compositions and methods of the present disclosure include a cationic lipid having the compound structure:

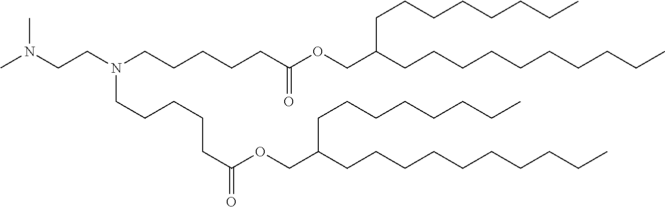 [SMILES: CCCCCCCCCCC(CCCCCCCC)COC(=O)CCCCCN(CCCCCC(=O)OCC(CCCCCCCC)CCCCCCCCCC)CCN(C)C]

and pharmaceutically acceptable salts thereof. In some embodiments, the compositions and methods of the present disclosure include a cationic lipid having the compound structure:

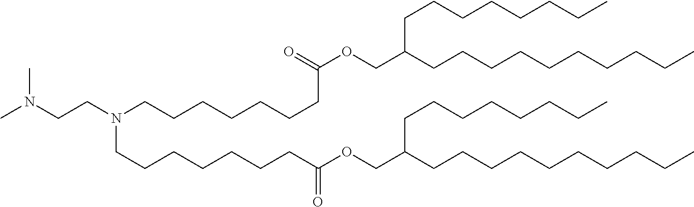 [SMILES: CCCCCCCCCCC(CCCCCCCC)COC(=O)CCCCCCCN(CCCCCCCC(=O)OCC(CCCCCCCC)CCCCCCCCCC)CCN(C)C]

and pharmaceutically acceptable salts thereof. In some embodiments, the compositions and methods of the present disclosure include a cationic lipid having the compound structure:

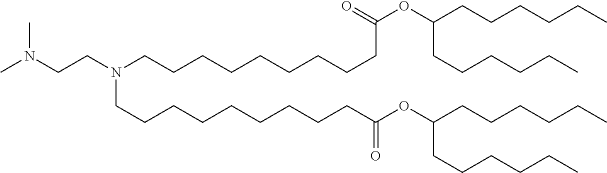 [SMILES: CCCCCCC(CCCCCC)OC(=O)CCCCCCCCCN(CCCCCCCCCC(=O)OC(CCCCCC)CCCCCC)CCN(C)C]

and pharmaceutically acceptable salts thereof. In some embodiments, the compositions and methods of the present disclosure include a cationic lipid having the compound structure:

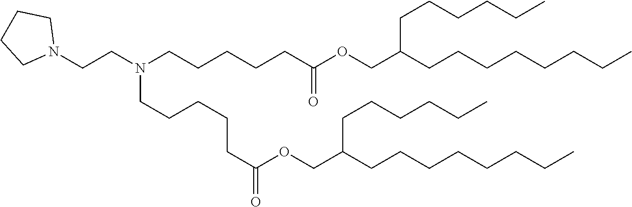 [SMILES: CCCCCCCCC(CCCCCC)COC(=O)CCCCCN(CCCCCC(=O)OCC(CCCCCC)CCCCCCCC)CCN1CCCC1]

and pharmaceutically acceptable salts thereof. In some embodiments, the compositions and methods of the present disclosure include a cationic lipid having the compound structure:

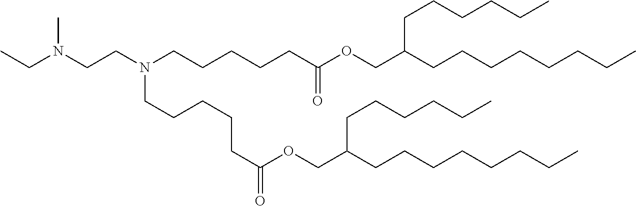 [SMILES: CCCCCCCCC(CCCCCC)COC(=O)CCCCCN(CCCCCC(=O)OCC(CCCCCC)CCCCCCCC)CCN(C)CC]

and pharmaceutically acceptable salts thereof. In some embodiments, the compositions and methods of the present disclosure include a cationic lipid having the compound structure:

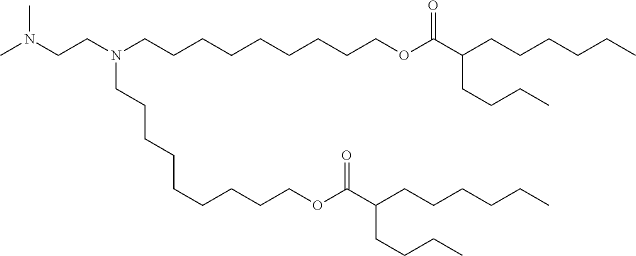 [SMILES: CCCCCCC(CCCC)C(=O)OCCCCCCCCCN(CCCCCCCCCOC(=O)C(CCCC)CCCCCC)CCN(C)C]

and pharmaceutically acceptable salts thereof.

Other suitable cationic lipids for use in the compositions and methods of the disclosure include the cationic lipids as described in International Patent Publication WO 2017/004143, which is incorporated herein by reference. In some embodiments, the compositions and methods of the present disclosure include a cationic lipid having the compound structure:

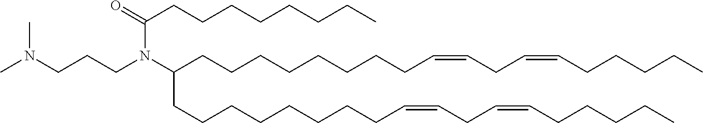 [SMILES: CCCCC/C=C\C/C=C\CCCCCCCCC(CCCCCCCC/C=C\C/C=C\CCCCC)N(CCCN(C)C)C(=O)CCCCCCCC]

and pharmaceutically acceptable salts thereof. In some embodiments, the compositions and methods of the present disclosure include a cationic lipid having the compound structure:

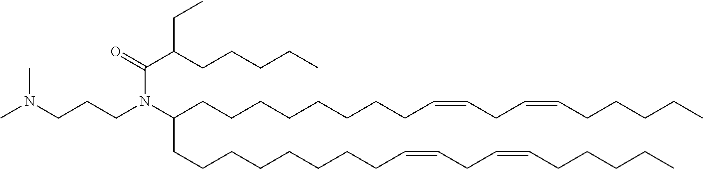 [SMILES: CCCCC/C=C\C/C=C\CCCCCCCCC(CCCCCCCC/C=C\C/C=C\CCCCC)N(CCCN(C)C)C(=O)C(CC)CCCCC]

and pharmaceutically acceptable salts thereof. In some embodiments, the compositions and methods of the present disclosure include a cationic lipid having the compound structure:

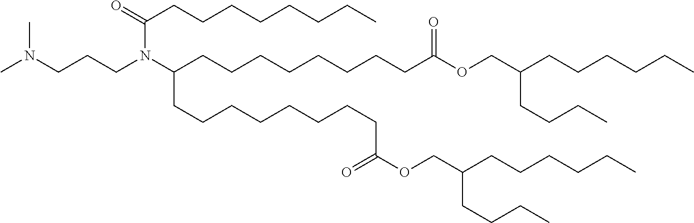 [SMILES: CCCCCCCCC(=O)N(CCCN(C)C)C(CCCCCCCCC(=O)OCC(CCCC)CCCCCC)CCCCCCCCC(=O)OCC(CCCC)CCCCCC]

and pharmaceutically acceptable salts thereof. In some embodiments, the compositions and methods of the present disclosure include a cationic lipid having the compound structure:

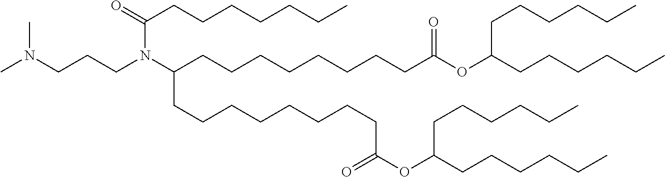 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC]

and pharmaceutically acceptable salts thereof. In some embodiments, the compositions and methods of the present disclosure include a cationic lipid having the compound structure:

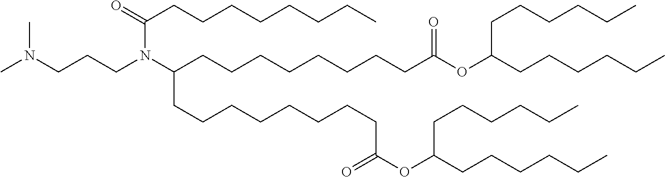 [SMILES: CCCCCCCCC(=O)N(CCCN(C)C)C(CCCCCCCCC(=O)OC(CCCCCC)CCCCCC)CCCCCCCCC(=O)OC(CCCCCC)CCCCCC]

and pharmaceutically acceptable salts thereof. In some embodiments, the compositions and methods of the present disclosure include a cationic lipid having the compound structure:

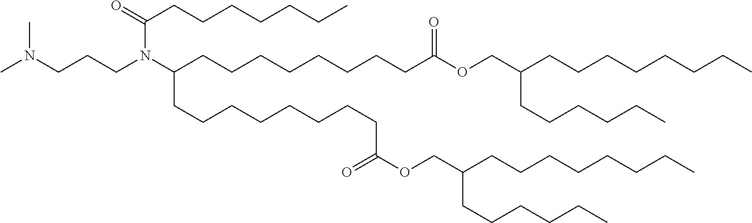 [SMILES: CCCCCCCCC(CCCCCC)COC(=O)CCCCCCCCC(CCCCCCCCC(=O)OCC(CCCCCC)CCCCCCCC)N(CCCN(C)C)C(=O)CCCCCCC]

and pharmaceutically acceptable salts thereof. In some embodiments, the compositions and methods of the present disclosure include a cationic lipid having the compound structure:

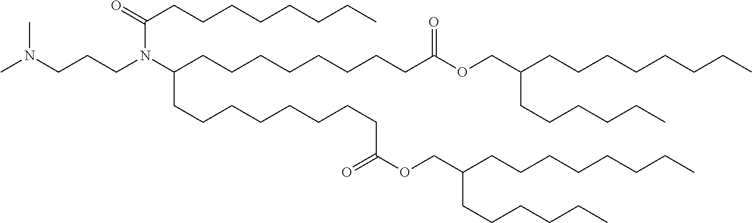 [SMILES: CCCCCCCCC(=O)N(CCCN(C)C)C(CCCCCCCCC(=O)OCC(CCCCCC)CCCCCCCC)CCCCCCCCC(=O)OCC(CCCCCC)CCCCCCCC]

and pharmaceutically acceptable salts thereof. In some embodiments, the compositions and methods of the present disclosure include a cationic lipid having the compound structure:

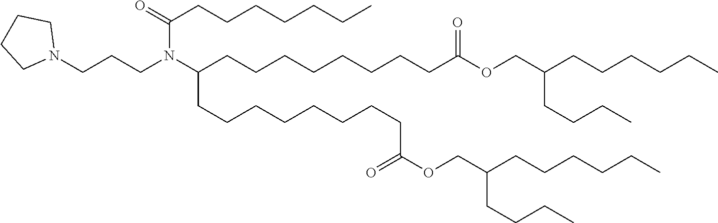 [SMILES: CCCCCCCC(=O)N(CCCN1CCCC1)C(CCCCCCCCC(=O)OCC(CCCC)CCCCCC)CCCCCCCCC(=O)OCC(CCCC)CCCCCC]

and pharmaceutically acceptable salts thereof. In some embodiments, the compositions and methods of the present disclosure include a cationic lipid having the compound structure:

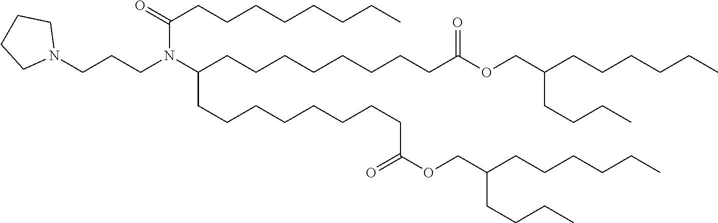 [SMILES: CCCCCCCCC(=O)N(CCCN1CCCC1)C(CCCCCCCCC(=O)OCC(CCCC)CCCCCC)CCCCCCCCC(=O)OCC(CCCC)CCCCCC]

and pharmaceutically acceptable salts thereof. In some embodiments, the compositions and methods of the present disclosure include a cationic lipid having the compound structure:

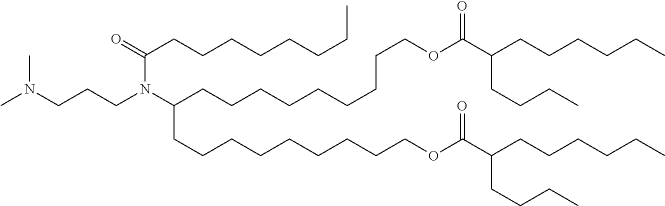 [SMILES: CCCCCCCCC(=O)N(CCCN(C)C)C(CCCCCCCCCOC(=O)C(CCCC)CCCCCC)CCCCCCCCCOC(=O)C(CCCC)CCCCCC]

and pharmaceutically acceptable salts thereof. In some embodiments, the compositions and methods of the present disclosure include a cationic lipid having the compound structure:

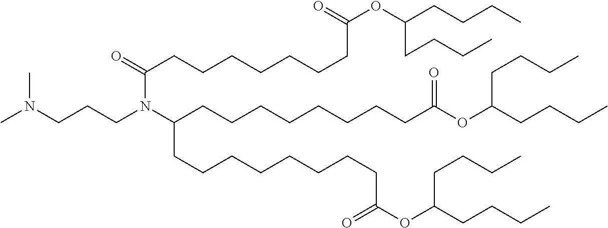 [SMILES: CCCCC(CCCC)OC(=O)CCCCCCCCC(CCCCCCCCC(=O)OC(CCCC)CCCC)N(CCCN(C)C)C(=O)CCCCCCCC(=O)OC(CCCC)CCCC]

and pharmaceutically acceptable salts thereof. In some embodiments, the compositions and methods of the present disclosure include a cationic lipid having the compound structure:

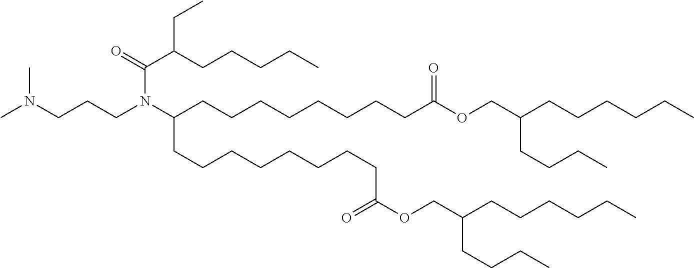 [SMILES: CCCCCCC(CCCC)COC(=O)CCCCCCCCC(CCCCCCCCC(=O)OCC(CCCC)CCCCCC)N(CCCN(C)C)C(=O)C(CC)CCCCC]

and pharmaceutically acceptable salts thereof. In some embodiments, the compositions and methods of the present disclosure include a cationic lipid having the compound structure:

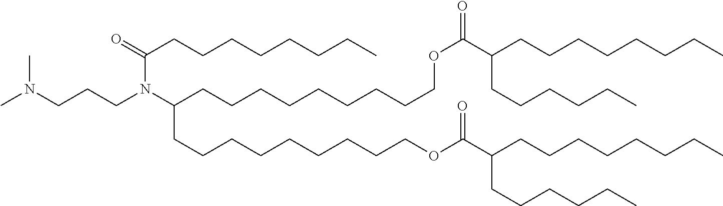 [SMILES: CCCCCCCCC(=O)N(CCCN(C)C)C(CCCCCCCCCOC(=O)C(CCCCCC)CCCCCCCC)CCCCCCCCCOC(=O)C(CCCCCC)CCCCCCCC]

and pharmaceutically acceptable salts thereof. In some embodiments, the compositions and methods of the present disclosure include a cationic lipid having the compound structure:

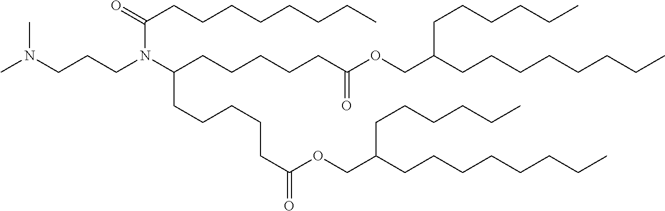 [SMILES: CCCCCCCCC(=O)N(CCCN(C)C)C(CCCCCC(=O)OCC(CCCCCC)CCCCCCCC)CCCCCC(=O)OCC(CCCCCC)CCCCCCCC]

and pharmaceutically acceptable salts thereof. In some embodiments, the compositions and methods of the present disclosure include a cationic lipid having the compound structure:

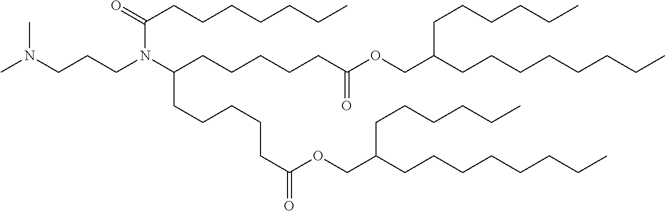 [SMILES: CCCCCCCCC(CCCCCC)COC(=O)CCCCCC(CCCCCC(=O)OCC(CCCCCC)CCCCCCCC)N(CCCN(C)C)C(=O)CCCCCCC]

and pharmaceutically acceptable salts thereof. In some embodiments, the compositions and methods of the present disclosure include a cationic lipid having the compound structure:

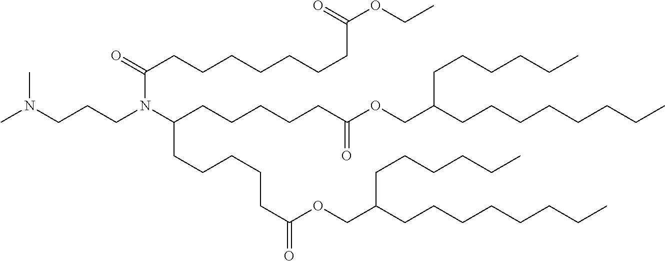 [SMILES: CCCCCCCCC(CCCCCC)COC(=O)CCCCCC(CCCCCC(=O)OCC(CCCCCC)CCCCCCCC)N(CCCN(C)C)C(=O)CCCCCCCC(=O)OCC]

and pharmaceutically acceptable salts thereof. In some embodiments, the compositions and methods of the present disclosure include a cationic lipid having the compound structure:

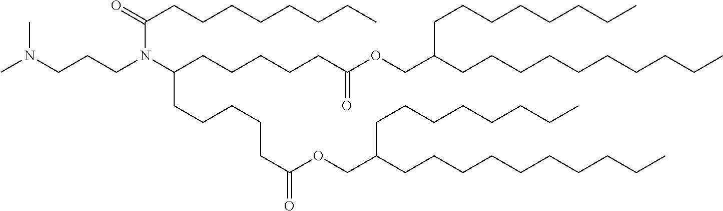 [SMILES: CCCCCCCCCCC(CCCCCCCC)COC(=O)CCCCCC(CCCCCC(=O)OCC(CCCCCCCC)CCCCCCCCCC)N(CCCN(C)C)C(=O)CCCCCCCC]

and pharmaceutically acceptable salts thereof.

In some embodiments, lipid nanoparticles of the present disclosure comprise a cationic lipid selected from DOTAP (1,2-dioleyl-3-trimethylammonium propane), DODAP (1,2-dioleyl-3-dimethylammonium propane), DOTMA (1,2-di-O-octadecenyl-3-trimethylammonium propane), DLinKC2DMA, DLin-KC2-DM, C12-200, cKK-E12 (3,6-bi s(4-(bis(2-hydroxydodecyl)amino)butyl)piperazine-2,5-dione), HGT5000, HGT5001, HGT4003, ICE, OF-02 and combinations thereof.

Other suitable cationic lipids for use in the compositions and methods of the disclosure include the cationic lipids as described in International Patent Publication WO 2017/075531, which is incorporated herein by reference. In some embodiments, the compositions and methods of the present disclosure include a cationic lipid of the following formula:

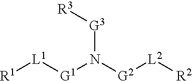

or a pharmaceutically acceptable salt thereof, wherein one of L¹ or L² is —O(C═O)—, —(C═O)O—, —C(═O)—, —O—, —S(O)ₓ, —S—S—, —C(═O)S—, —SC(═O)—, —NR^{a}C(═O)—, —C(═O)NR^{a}—, NR^{a}C(═O)NR^{a}—, —OC(═O)NR^{a}—, or —NR^{a}C(═O)O—; and the other of L¹ or L² is —O(C═O)—, —(C═O)O—, —C(═O)—, —O—, —S(O)ₓ, —S—S—, —C(═O)S—, SC(═O)—, —NR^{a}C(═O)—, —C(═O)NR^{a}—, NR^{a}C(═O)NR^{a}—, —OC(═O)NR^{a}— or —NR^{a}C(═O)O— or a direct bond; G¹ and G² are each independently unsubstituted C₁-C₁₂ alkylene or C₁-C₁₂ alkenylene; G³ is C₁-C₂₄ alkylene, C₁-C₂₄ alkenylene, C₃-C₈ cycloalkylene, C₃-C₈ cycloalkenylene; IV is H or C₁-C₁₂ alkyl; R¹ and R² are each independently C₆-C₂₄ alkyl or C₆-C₂₄ alkenyl; R³ is H, OR⁵, CN, —C(═O)OR⁴, —OC(═O)R⁴ or —NR⁵C(═O)R⁴; R⁴ is C₁-C₁₂ alkyl; R⁵ is H or C₁-C₆ alkyl; and x is 0, 1 or 2.

Other suitable cationic lipids for use in the compositions and methods of the disclosure include the cationic lipids as described in International Patent Publication WO 2017/117528, which is incorporated herein by reference. In some embodiments, the compositions and methods of the present disclosure include a cationic lipid having the compound structure:

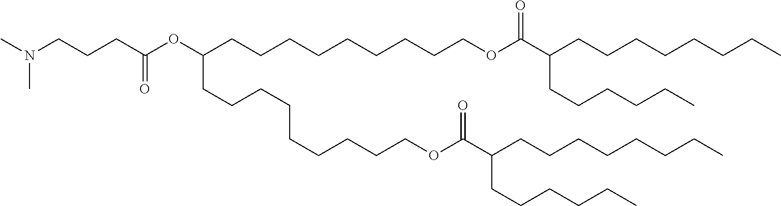 [SMILES: CCCCCCCCC(CCCCCC)C(=O)OCCCCCCCCCC(CCCCCCCCCOC(=O)C(CCCCCC)CCCCCCCC)OC(=O)CCCN(C)C]

and pharmaceutically acceptable salts thereof. In some embodiments, the compositions and methods of the present disclosure include a cationic lipid having the compound structure:

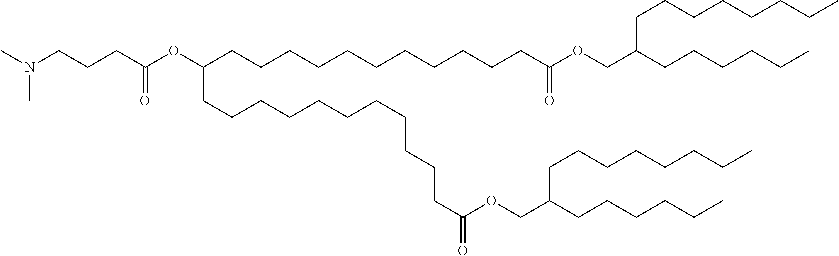 [SMILES: CCCCCCCCC(CCCCCC)COC(=O)CCCCCCCCCCCC(CCCCCCCCCCCC(=O)OCC(CCCCCC)CCCCCCCC)OC(=O)CCCN(C)C]

and pharmaceutically acceptable salts thereof. In some embodiments, the compositions and methods of the present disclosure include a cationic lipid having the compound structure:

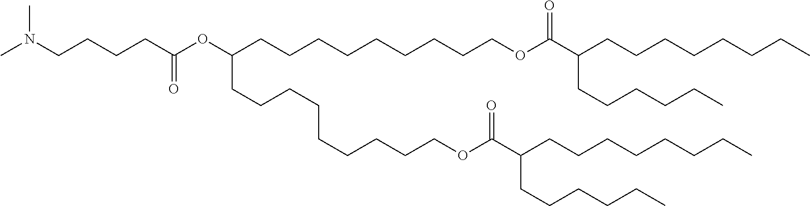 [SMILES: CCCCCCCCC(CCCCCC)C(=O)OCCCCCCCCCC(CCCCCCCCCOC(=O)C(CCCCCC)CCCCCCCC)OC(=O)CCCCN(C)C]

and pharmaceutically acceptable salts thereof.

Other suitable cationic lipids for use in the compositions and methods of the disclosure include the cationic lipids as described in International Patent Publication WO 2017/049245, which is incorporated herein by reference. In some embodiments, the cationic lipids of the compositions and methods of the present disclosure include a compound of one of the following formulas:

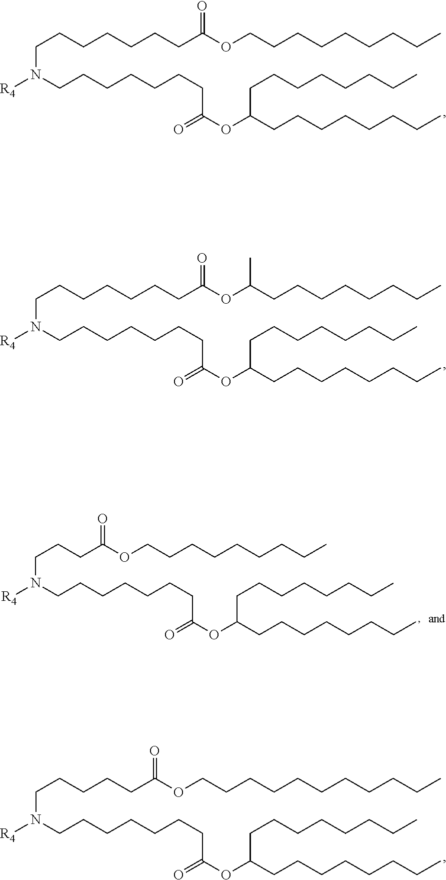

and pharmaceutically acceptable salts thereof. For any one of these four formulas, R₄ is independently selected from —(CH₂)ₙQ and —(CH₂)ₙCHQR; Q is selected from the group consisting of —OR, —OH, —O(CH₂)ₙN(R)₂, —OC(O)R, —CX₃, —CN, —N(R)C(O)R, —N(H)C(O)R, —N(R)S(O)₂R, —N(H)S(O)₂R, —N(R)C(O)N(R)₂, —N(H)C(O)N(R)₂, —N(H)C(O)N(H)(R), —N(R)C(S)N(R)₂, —N(H)C(S)N(R)₂, —N(H)C(S)N(H)(R), and a heterocycle; R is independently selected from the group consisting of C₁₋₃ alkyl, C₂₋₃ alkenyl, and H; and n is 1, 2, or 3. In certain embodiments, the compositions and methods of the present disclosure include a cationic lipid having a compound structure of:

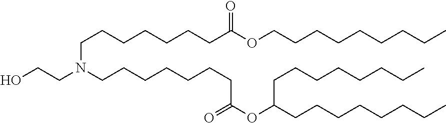 [SMILES: CCCCCCCCCOC(=O)CCCCCCCN(CCO)CCCCCCCC(=O)OC(CCCCCCCC)CCCCCCCC]

and pharmaceutically acceptable salts thereof. In certain embodiments, the compositions and methods of the present disclosure include a cationic lipid having a compound structure of:

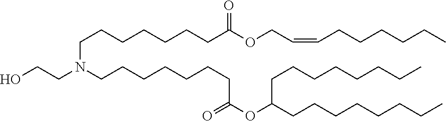 [SMILES: CCCCCC/C=C\COC(=O)CCCCCCCN(CCO)CCCCCCCC(=O)OC(CCCCCCCC)CCCCCCCC]

and pharmaceutically acceptable salts thereof. In certain embodiments, the compositions and methods of the present disclosure include a cationic lipid having a compound structure of:

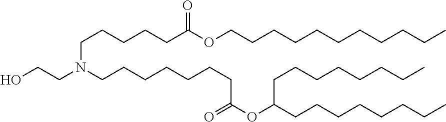 [SMILES: CCCCCCCCCCCOC(=O)CCCCCN(CCO)CCCCCCCC(=O)OC(CCCCCCCC)CCCCCCCC]

and pharmaceutically acceptable salts thereof. In certain embodiments, the compositions and methods of the present disclosure include a cationic lipid having a compound structure of:

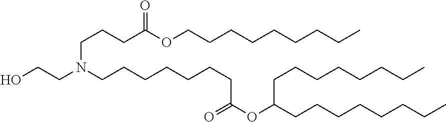 [SMILES: CCCCCCCCCOC(=O)CCCN(CCO)CCCCCCCC(=O)OC(CCCCCCCC)CCCCCCCC]

and pharmaceutically acceptable salts thereof.

Other suitable cationic lipids for use in the compositions and methods of the disclosure include the cationic lipids as described in International Patent Publication WO 2017/173054 and WO 2015/095340, each of which is incorporated herein by reference. In certain embodiments, the compositions and methods of the present disclosure include a cationic lipid having a compound structure of:

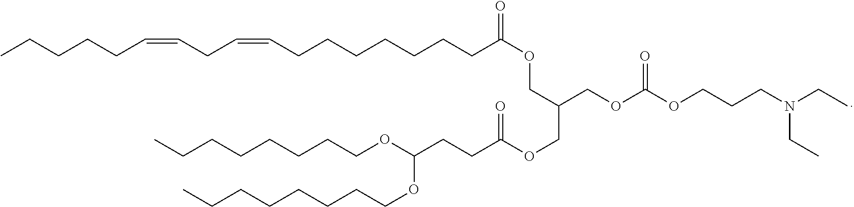 [SMILES: CCCCC/C=C\C/C=C\CCCCCCCC(=O)OCC(COC(=O)CCC(OCCCCCCCC)OCCCCCCCC)COC(=O)OCCCN(CC)CC]

and pharmaceutically acceptable salts thereof

In certain embodiments, the compositions and methods of the present disclosure include a cationic lipid having a compound structure of:

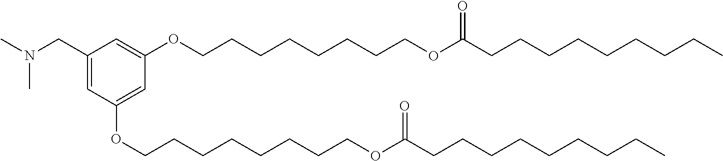 [SMILES: CCCCCCCCCC(=O)OCCCCCCCCOc1cc(CN(C)C)cc(OCCCCCCCCOC(=O)CCCCCCCCC)c1]

and pharmaceutically acceptable salts thereof. In certain embodiments, the compositions and methods of the present disclosure include a cationic lipid having a compound structure of:

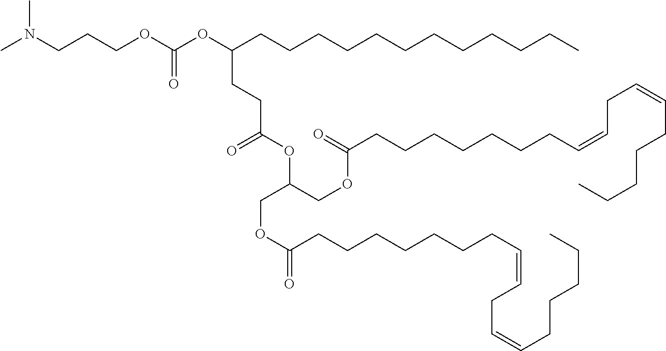 [SMILES: CCCCC/C=C\C/C=C\CCCCCCCC(=O)OCC(COC(=O)CCCCCCC/C=C\C/C=C\CCCCC)OC(=O)CCC(CCCCCCCCCCCC)OC(=O)OCCCN(C)C]

and pharmaceutically acceptable salts thereof. In certain embodiments, the compositions and methods of the present disclosure include a cationic lipid having a compound structure of:

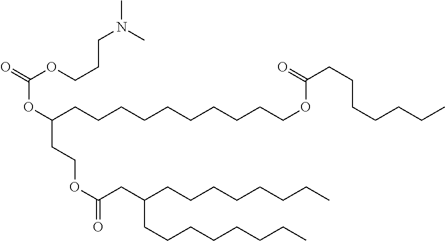 [SMILES: CCCCCCCCC(CCCCCCCC)CC(=O)OCCC(CCCCCCCCCCOC(=O)CCCCCCC)OC(=O)OCCCN(C)C]

and pharmaceutically acceptable salts thereof.

Other suitable cationic lipids for use in the compositions and methods of the disclosure include cholesterol-based cationic lipids. In certain embodiments, the compositions and methods of the present disclosure include imidazole cholesterol ester or “ICE”, having a compound structure of:

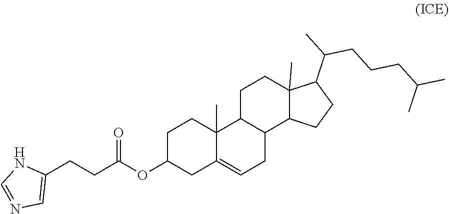 [SMILES: CC(C)CCCC(C)C1CCC2C3CC=C4CC(OC(=O)CCc5cnc[nH]5)CCC4(C)C3CCC12C]

and pharmaceutically acceptable salts thereof.

Other suitable cationic lipids for use in the compositions and methods of the present disclosure include cleavable cationic lipids as described in International Patent Publication WO 2012/170889, which is incorporated herein by reference. In some embodiments, the compositions and methods of the present disclosure include a cationic lipid of the following formula:

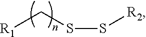

wherein R₁ is selected from the group consisting of imidazole, guanidinium, amino, imine, enamine, an optionally-substituted alkyl amino (e.g., an alkyl amino such as dimethylamino) and pyridyl; wherein R₂ is selected from the group consisting of one of the following two formulas:

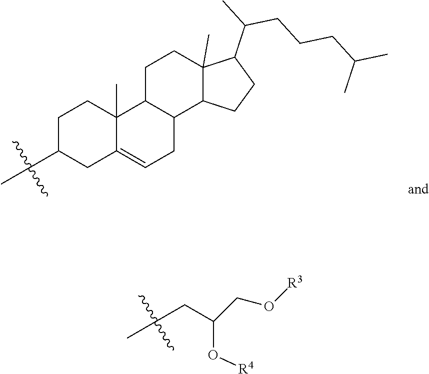

and wherein R₃ and R₄ are each independently selected from the group consisting of an optionally substituted, variably saturated or unsaturated C₆-C₂₀ alkyl and an optionally substituted, variably saturated or unsaturated C₆-C₂₀ acyl; and wherein n is zero or any positive integer (e.g., one, two, three, four, five, six, seven, eight, nine, ten, eleven, twelve, thirteen, fourteen, fifteen, sixteen, seventeen, eighteen, nineteen, twenty or more). In certain embodiments, the compositions and methods of the present disclosure include a cationic lipid, “HGT4001”, having a compound structure of:

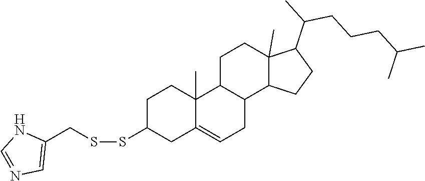 [SMILES: CC(C)CCCC(C)C1CCC2C3CC=C4CC(SSCc5cnc[nH]5)CCC4(C)C3CCC12C]

### (HGT4001)

and pharmaceutically acceptable salts thereof. In certain embodiments, the compositions and methods of the present disclosure include a cationic lipid, “HGT4002”, having a compound structure of:

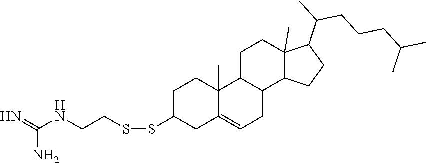 [SMILES: CC(C)CCCC(C)C1CCC2C3CC=C4CC(SSCCNC(=N)N)CCC4(C)C3CCC12C]

### (HGT4002)

and pharmaceutically acceptable salts thereof. In certain embodiments, the compositions and methods of the present disclosure include a cationic lipid, “HGT4003”, having a compound structure of:

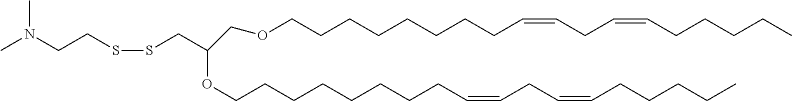 [SMILES: CCCCC/C=C\C/C=C\CCCCCCCCOCC(CSSCCN(C)C)OCCCCCCCC/C=C\C/C=C\CCCCC]

### (HGT4003)

and pharmaceutically acceptable salts thereof. In certain embodiments, the compositions and methods of the present disclosure include a cationic lipid, “HGT4004”, having a compound structure of:

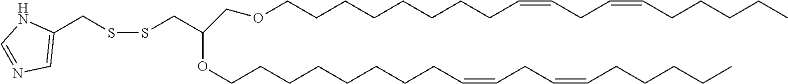 [SMILES: CCCCC/C=C\C/C=C\CCCCCCCCOCC(CSSCc1cnc[nH]1)OCCCCCCCC/C=C\C/C=C\CCCCC]

### (HGT4004)

and pharmaceutically acceptable salts thereof. In certain embodiments, the compositions and methods of the present disclosure include a cationic lipid “HGT4005”, having a compound structure of:

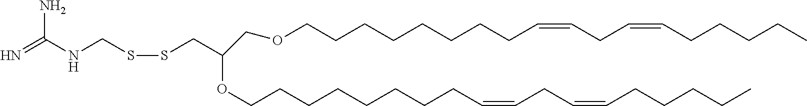 [SMILES: CCCCC/C=C\C/C=C\CCCCCCCCOCC(CSSCNC(=N)N)OCCCCCCCC/C=C\C/C=C\CCCCC]

### (HGT4005)

and pharmaceutically acceptable salts thereof.

In some embodiments, cationic lipids have the structure:

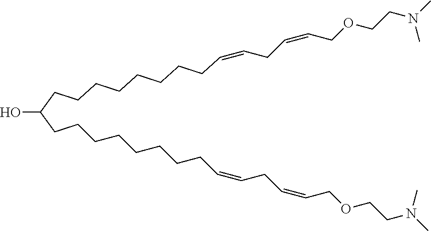 [SMILES: CN(C)CCOC/C=C\C/C=C\CCCCCCCCC(O)CCCCCCCC/C=C\C/C=C\COCCN(C)C]

or a pharmaceutically acceptable salt thereof.

In some embodiments, cationic lipids have the structure:

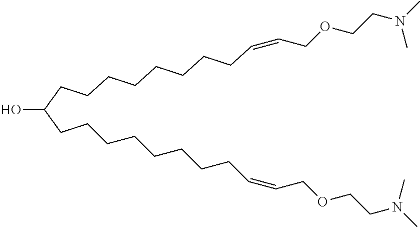 [SMILES: CN(C)CCOC/C=C\CCCCCCCCC(O)CCCCCCCC/C=C\COCCN(C)C]

or a pharmaceutically acceptable salt thereof.

In some embodiments, cationic lipids have the structure:

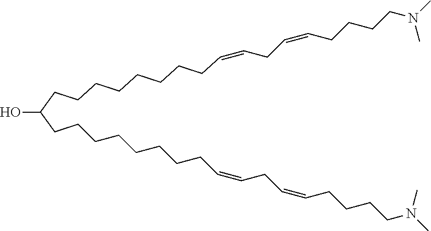 [SMILES: CN(C)CCCC/C=C\C/C=C\CCCCCCCCC(O)CCCCCCCC/C=C\C/C=C\CCCCN(C)C]

or a pharmaceutically acceptable salt thereof.

In some embodiments, cationic lipids have the structure:

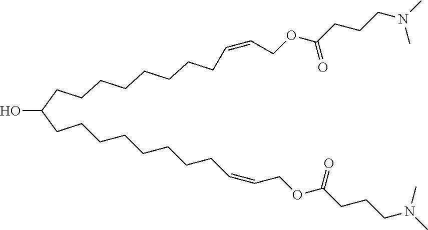 [SMILES: CN(C)CCCC(=O)OC/C=C\CCCCCCCCC(O)CCCCCCCC/C=C\COC(=O)CCCN(C)C]

or a pharmaceutically acceptable salt thereof.

In some embodiments, the one or more cationic lipids comprise cKK-E12 3,6-bis(4-(bis(2-hydroxydodecyl)amino)butyl)piperazine-2,5-dione):

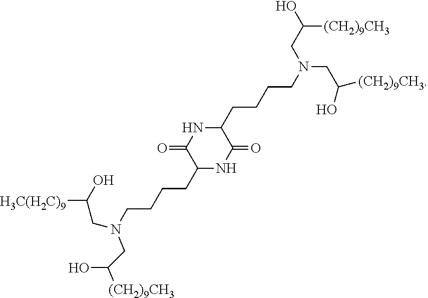

In some embodiments, cationic lipids have the structure:

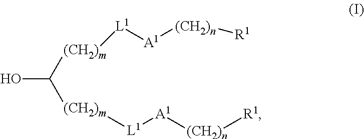

wherein:
each L¹ is independently C₂-C₁₂-alkenylene; each A¹ is independently a linker group that is a covalent bond, O, S, NH, S—S, an amide, an ester, a thioester, or an anhydride group; each R¹ is an ionizable nitrogen-containing group; each m is independently an integer of 6 to 20; and each n is independently an integer of 1 to 1.

In embodiments, each L¹ is independently C₂-C₁₂-alkenylene comprising one carbon-carbon double bond. In embodiments, each L¹ is independently C₆-C₁₂-alkenylene comprising one carbon-carbon double bond or two carbon-carbon double bonds. In embodiments, L¹ comprises one carbon-carbon double bond. In embodiments, L¹ comprises two carbon-carbon double bonds. In embodiments, each L¹ is independently —CH═CH—CH₂— or —CH═CH—CH₂—CH═CH—CH₂—. In embodiments, each L¹ is —CH═CH—CH₂—. In embodiments, each L¹ is —CH═CH—CH₂—CH═CH—CH₂—.

In some embodiments, a cationic lipid is N-[1-(2,3-dioleyloxy)propyl]-N,N,N-trimethylammonium chloride (“DOTMA”). (Feigner et al. (Proc. Nat'l Acad. Sci. 84, 7413 (1987); U.S. Pat. No. 4,897,355, which is incorporated herein by reference). DOTMA can be formulated alone or can be combined with a neutral lipid (e.g., dioleoylphosphatidyl-ethanolamine or “DOPE”) or still other cationic or non-cationic lipids into a liposomal transfer vehicle or a lipid nanoparticle, and such liposomes can be used to enhance the delivery of nucleic acids into target cells. Other cationic lipids suitable for the compositions and methods of the present invention include, for example, 5-carboxyspermylglycinedioctadecylamide (“DOGS”); 2,3-dioleyloxy-N-[2(spermine-carboxamido)ethyl]-N,N-dimethyl-1-propanaminium (“DOSPA”) (Behr et al. Proc. Nat.'l Acad. Sci. 86, 6982 (1989), U.S. Pat. Nos. 5,171,678; 5,334,761); 1,2-Dioleoyl-3-Dimethylammonium-Propane (“DODAP”); 1,2-Dioleoyl-3-Trimethylammonium-Propane (“DOTAP”). Additional exemplary cationic lipids suitable for the compositions and methods of the present invention also include: 1,2-distearyloxy-N,N-dimethyl-3-aminopropane (“DSDMA”); 1,2-dioleyloxy-N,N-dimethyl-3-aminopropane (“DODMA”); 1,2-dilinoleyloxy-N,N-dimethyl-3-aminopropane (“DLinDMA”); 1,2-dilinolenyloxy-N,N-dimethyl-3-aminopropane (“DLenDMA”); N-dioleyl-N,N-dimethylammonium chloride (“DODAC”); N,N-distearyl-N,N-dimethylarnrnonium bromide (“DDAB”); DLin-KC2-DM; C12-200; N-(1,2-dimyristyloxyprop-3-yl)-N,N-dimethyl-N-hydroxyethyl ammonium bromide (“DMRIE”); 3-dimethylamino-2-(cholest-5-en-3-beta-oxybutan-4-oxy)-1-(cis,cis-9,12-octadecadienoxy)propane (“CLinDMA”); 2-[5′-(cholest-5-en-3-beta-oxy)-3′-oxapentoxy)-3-dimethyl-1-(cis,cis-9′,1-2′-octadecadienoxy)propane (“CpLinDMA”); N,N-dimethyl-3,4-dioleyloxybenzylamine (“DMOBA”); 1,2-N,N′-dioleylcarbamyl-3-dimethylaminopropane (“DOcarbDAP”); 2,3-Dilinoleoyloxy-N,N-dimethylpropylamine (“DLinDAP”); 1,2-N,N′-Dilinoleylcarbamyl-3-dimethylaminopropane (“DLincarbDAP”); 1,2-Dilinoleoylcarbamyl dimethylaminopropane (“DLinCDAP”); 2,2-dilinoleyl-4-dimethylaminomethyl-[1,3]-dioxolane (“DLin-K-DMA”); 2-((8-[(3P)-cholest-5-en-3-yloxy]octyl)oxy)-N, N-dimethyl [(9Z,12Z)-octadeca-9,12-dien-1-yloxy]propane-1-amine (“Octyl-CLinDMA”); (2R)-2-((8-[(3beta)-cholest-5-en-3-yloxy]octyl)oxy)-N, N-dimethyl-3-[(9Z,12Z)-octadeca-9,12-dien yloxy]propan-1-amine (“Octyl-CLinDMA (2R)”); (2S)-2-((8-[(3P)-cholest-5-en yloxy]octyl)oxy)-N, fsl-dimethyh3-[(9Z,12Z)-octadeca-9,12-dien-1-yloxy]propan-1-amine (“Octyl-CLinDMA (2S)”); 2,2-dilinoleyl-4-dimethylaminoethyl-[1,3]-dioxolane (“DLin-K-XTC2-DMA”); and 2-(2,2-di((9Z,12Z)-octadeca-9,12-dien-1-yl)-1,3-dioxolan-4-yl)-N,N-dimethylethanamine (“DLin-KC2-DMA”) (see, WO 2010/042877, which is incorporated herein by reference; Semple et al., Nature Biotech. 28: 172-176 (2010)). (Heyes, J., et al., J Controlled Release 107: 276-287 (2005); Morrissey, D V., et al., Nat. Biotechnol. 23(8): 1003-1007 (2005); International Patent Publication WO 2005/121348). In some embodiments, one or more of the cationic lipids comprise at least one of an imidazole, dialkylamino, or guanidinium moiety.

In some embodiments, one or more of the cationic lipids comprise at least one of an imidazole, dialkylamino, or guainidium moiety. In some embodiments, one or more of the cationic lipids comprise include 2,2-Dilinoleyl-4-dimethylaminoethyl-[1,3]-dioxolane (“XTC”); (3aR,5s,6aS)—N,N-dimethyl-2,2-di((9Z,12Z)-octadeca-9,12-dienyl)tetrahydro-3aH-cyclopenta[d][1,3]dioxol-5-amine and/or 4,7,13-tris(3-oxo-3-(undecylamino)propyl)-N1,N16-diundecyl-4,7,10,13-tetraazahexadecane-1,16-diamide (“NC98-5”).

In some embodiments, the compositions of the present disclosure include one or more cationic lipids that constitute at least about 5%, 10%, 20%, 30%, 35%, 40%, 45%, 50%, 55%, 60%, 65%, or 70%, measured by weight, of the total lipid content in the composition, e.g., a lipid nanoparticle. In some embodiments, the compositions of the present disclosure include one or more cationic lipids that constitute at least about 5%, 10%, 20%, 30%, 35%, 40%, 45%, 50%, 55%, 60%, 65%, or 70%, measured as a mol %, of the total lipid content in the composition, e.g., a lipid nanoparticle. In some embodiments, the compositions of the present disclosure include one or more cationic lipids that constitute about 30-70% (e.g., about 30-65%, about 30-60%, about 30-55%, about 30-50%, about 30-45%, about 30-40%, about 35-50%, about 35-45%, or about 35-40%), measured by weight, of the total lipid content in the composition, e.g., a lipid nanoparticle. In some embodiments, the compositions of the present disclosure include one or more cationic lipids that constitute about 30-70% (e.g., about 30-65%, about 30-60%, about 30-55%, about 30-50%, about 30-45%, about 30-40%, about 35-50%, about 35-45%, or about 35-40%), measured as mol %, of the total lipid content in the composition, e.g., a lipid nanoparticle.

In some embodiments, sterol-based cationic lipids may be use instead or in addition to cationic lipids described herein. Suitable sterol-based cationic lipids are dialkylamino-, imidazole-, and guanidinium-containing sterol-based cationic lipids. For example, certain embodiments are directed to a composition comprising one or more sterol-based cationic lipids comprising an imidazole, for example, the imidazole cholesterol ester or “ICE” lipid (3S,10R, 13R, 17R)-10,13-dimethyl-17-((R)-6-methylheptan-2-yl)-2, 3, 4, 7, 8, 9, 10, 11, 12, 13, 14, 15, 16,17-tetradecahydro-1H-cyclopenta[a]phenanthren-3-yl 3-(1H-imidazol-4-yl)propanoate, as represented by structure (I) below. In certain embodiments, a lipid nanoparticle may comprise one or more imidazole-based cationic lipids, for example, the imidazole cholesterol ester or “ICE” lipid (3S,10R, 13R, 17R)-10,13-dimethyl-17-((R)-6-methylheptan-2-yl)-2, 3, 4, 7, 8, 9, 10, 11, 12, 13, 14, 15, 16,17-tetradecahydro-1H-cyclopenta[a]phenanthren-3-yl3-(1H-imidazol-4-yl)propanoate, as represented by the following structure:

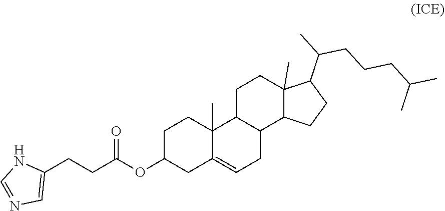 [SMILES: CC(C)CCCC(C)C1CCC2C3CC=C4CC(OC(=O)CCc5cnc[nH]5)CCC4(C)C3CCC12C]

In some embodiments, the percentage of cationic lipid in a lipid nanoparticle may be greater than 30-80% of the lipid nanoparticles by molar ratio. In some embodiments, the cationic lipid (e.g., ICE lipid) constitutes about 30%, about 35%, about 40%, about 45%, about 50%, about 55%, about 60%, about 65%, about 70%, about 75%, or about 80% of the lipid nanoparticles by molar ratio.

d. Non-Cationic Lipids

In some embodiments, a particle of the present disclosure includes a non-cationic lipid. Non-cationic lipids are any neutral, zwitterionic, or anionic lipid. The non-cationic lipid may be any appropriate non-cationic lipid for particle, e.g., nanoparticle, e.g., lipid nanoparticle, formulation. For example, the non-cationic lipid may be an anionic lipid or a neutral lipid.

An anionic lipid is any of a number of lipid species that carry a net negative charge at a selected pH, such as physiological pH. A neutral lipid is any of a number of lipid species that carry a net neutral charge at a selected pH, such as physiological pH. A zwitterionic lipid is any of a number of lipid species that carry both positive and negative charges, but whose total charge is not positive (may be either neutral or negative). Anionic lipids suitable for use in particles of the present disclosure include, but are not limited to, phosphatidylglycerol, cardiolipin, diacylphosphatidylserine, diacylphosphatidic acid, N-dodecanoyl phosphatidylethanoloamine, N-succinyl phosphatidylethanolamine, N-glutaryl phosphatidylethanolamine, lysylphosphatidylglycerol, and other anionic modifying groups joined to neutral lipids. In some embodiments, the anionic lipid is 1,2-dioleoyl-sn-glycero phospho-(1′-rac-glycerol).

A neutral lipid generally refers to a lipid which exists either in an uncharged or neutral zwitterionic form at physiological pH. Such lipids include, for example diacylphosphatidylcholine, diacylphosphatidylethanolamine, ceramide, sphingomyelin, dihydrosphingomyelin, cephalin, and cerebrosides. The selection of neutral lipids for use in the particles described herein is generally guided by consideration of, e.g., nanoparticle and stability of the nanoparticles in the bloodstream. In some embodiments, the neutral lipid component is a lipid having two acyl groups, (e.g., diacylphosphatidylcholine and diacylphosphatidylethanolamine). Lipids having a variety of acyl chain groups of varying chain length and degree of saturation are available or may be isolated or synthesized by well-known techniques. In some embodiments, lipids contain saturated fatty acids with carbon chain lengths in the range of C10 to C20. In some embodiments, lipids with mono or diunsaturated fatty acids with carbon chain lengths in the range of C10 to C20 may be used. Additionally, lipids having mixtures of saturated and unsaturated fatty acid chains can be used. For example, a neutral lipid for use in accordance with the present disclosure is DOPE, DSPC, POPC, DPPC or any related phosphatidylcholine. In some embodiments, a neutral lipid may be composed of sphingomyelin, dihydrosphingomyeline, or phospholipids with other head groups, such as serine and inositol. In some embodiments, the neutral lipid is 1,2-dioleoyl-sn-glycero-3-phosphatidylcholine.

In some embodiments, exemplary non-cationic lipids include, but are not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), 16-O-monomethyl PE, 16-O-dimethyl PE, 18-1-trans PE, 1-stearoyl-2-oleoyl-phosphatidyethanolamine (SOPE), cholesterol, phosphatidylglycerols, cardiolipins, diacylphosphatidylserines, diacylphosphatidic acids, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and other anionic modifying groups joined to neutral lipids or a mixture thereof. In some embodiments, the anionic lipid is 1,2-dioleoyl-sn-glycero-3-phospho-(1′-rac-glycerol). In some embodiments, the neutral lipid is 1,2-dioleoyl-sn-glycero-3-phosphatidylcholine.

In some embodiments, lipid nanoparticles of the present disclosure comprise a non-cationic lipid selected from DSPC (1,2-distearoyl-sn-glycero-3-phosphocholine), DPPC (1,2-dipalmitoyl-sn-glycero-3-phosphocholine), DOPE (1,2-dioleyl-sn-glycero-3-phosphoethanolamine), DOPC (1,2-dioleyl-sn-glycero-3-phosphotidylcholine) DPPE (1,2-dipalmitoyl-sn-glycero-3-phosphoethanolamine), DMPE (1,2-dimyristoyl-sn-glycero-3-phosphoethanolamine), DOPG (1,2-dioleoyl-sn-glycero-3-phospho-(1′-rac-glycerol)) or combinations thereof.

e. Aggregation Prevention Lipid

Lipid nanoparticles of the present disclosure may include a lipid that inhibits and/or reduces aggregation of particles during formation. Examples of lipids that reduce aggregation of particles during formation include polyethylene glycol (PEG)-modified lipids, monosialoganglioside Gm1, and polyamide oligomers (“PAO”) such as (described in U.S. Pat. No. 6,320,017). Other compounds with uncharged, hydrophilic, steric-barrier moieties, which prevent aggregation during formulation, like PEG, Gm1 or ATTA, can also be coupled to lipids for use as in accordance with the disclosure. Exemplary ATTA-lipids are described, e.g., in U.S. Pat. No. 6,320,017, and exemplary PEG-lipid conjugates are described, e.g., in U.S. Pat. Nos. 5,820,873, 5,534,499 and 5,885,613.

In some embodiments, a lipid is conjugated to the lipid nanoparticle of the present claims. This conjugated lipid may prevent aggregation of lipid nanoparticles in a formulation of the present disclosure. In some embodiments, the conjugated lipid is a PEG lipid. In some embodiments, the PEG lipid is a PEG-diacylglycerol (DAG); a PEG-dialkyloxypropyl (DAA); a PEG-phospholipid; a PEG-ceramide (Cer); or a mixture thereof. As a non-limiting example, PLGA may be conjugated to a lipid-terminating PEG forming PLGA-DSPE-PEG; PEG lipid is selected from PEG-c-DOMG and 1,2-Dimyristoyl-sn-glycerol; methoxypolyethylene Glycol (PEG-DMG); 1,2-Dimyristoyl-sn-glycerol; methoxypolyethylene Glycol 2000 (DMG-PEG2K) 1,2-Distearoyl-sn-glycerol; methoxypolyethylene Glycol (PEG-DSG); PEG-c-DOM; 1,2-Distearoyl-sn-glycerol; methoxypolyethylene glycol (PEG-DSG) 1,2-Dipalmitoyl-sn-glycerol; methoxypolyethylene glycol (PEG-DPG); PEG-lipid conjugates such as, e.g., PEG coupled to dialkyloxypropyls (e.g., PEG-DAA conjugates); PEG coupled to diacylglycerols (e.g., PEG-DAG conjugates); PEG coupled to cholesterol; PEG coupled to phosphatidylethanolamines; and PEG conjugated to ceramides; cationic PEG lipids; polyoxazoline (POZ)-lipid conjugates; polyamide oligomers (e.g., ATTA-lipid conjugates); and mixtures thereof. In some embodiments, the PEG is a PEG-dilauryloxypropyl (C12); a PEG-dimyristyloxypropyl (C14); a PEG-dipalmityloxypropyl (C16); a PEG-distearyloxypropyl (C18); PEG-c-DOMG, PEG-DMG, or a mixture thereof.

In some embodiments, lipid nanoparticles comprise ICE, DOPE, and DMG-PEG2K.

In some embodiments, the conjugated lipid that prevents aggregation of particles may be from 0% to about 20%, e.g., about 1% to about 15%, e.g., about 2% of the total lipid present in the particle (by mole percent of lipids). In some embodiments, the nucleic acid-lipid particle further includes cholesterol at, e.g., about 10 mol percent to about 60 mol percent or about 48 mol percent of the total lipid present in the particle.

In some embodiments, the conjugated PEG lipid is coupled to the surface of the lipid nanoparticle. In some embodiments, the PEG lipid is coupled to the surface of the lipid nanoparticle by an oxime linkage. In some embodiments, the conjugated PEG lipid is susceptible to decomposition in an acidic environment.

As non-limiting examples, a suitable lipid nanoparticle formulation may include a combination selected from cKK-E12, DOPE, cholesterol and DMG-PEG2K; C12-200, DOPE, cholesterol and DMG-PEG2K; HGT4003, DOPE, cholesterol and DMG-PEG2K; or ICE, DOPE, cholesterol and DMG-PEG2K or ICE, DOPE and DMG-PEG2K.

Additional combinations of lipids are described in the art, e.g., U.S. Ser. No. 62/420,421 (filed on Nov. 10, 2016), U.S. Ser. No. 62/421,021 (filed on Nov. 11, 2016), U.S. Ser. No. 62/464,327 (filed on Feb. 27, 2017), and PCT Application entitled “Novel ICE-based Lipid Nanoparticle Formulation for Delivery of mRNA,” filed on Nov. 10, 2017, the disclosures of which are included here in their full scope by reference.

f. Definition of Certain Chemical Terms

Alkynyl: As used herein, alkynyl refers to any hydrocarbon chain of either linear or branched configuration, having one or more carbon-carbon triple bonds occurring in any stable point along the chain, e.g. C2-C20 alkynyl refers to an alkynyl group having 2-20 carbons. Examples of an alkynyl group include prop-2-ynyl, but-2-ynyl, but ynyl, pent-2-ynyl, 3-methylpent-4-ynyl, hex-2-ynyl, hex-5-ynyl, etc. In embodiments, an alkynyl comprises one carbon-carbon triple bond. An alkynyl group may be unsubstituted or substituted with one or more substituent groups as described herein. For example, an alkynyl group may be substituted with one or more (e.g., 1, 2, 3, 4, 5, or 6 independently selected substituents) of halogen, —COR′, —CO₂H, —CO2R′, —CN, —OH, —OR′, —OCOR′, —OCO₂R′, —NH₂, —NHR′, —N(R′)₂, —SR′ or —SO₂R′, wherein each instance of R′ independently is C₁-C₂₀ aliphatic (e.g., C₁-C₂₀ alkyl, C₁-C₁₅ alkyl, C₁-C₁₀ alkyl, or C₁-C₃ alkyl). In embodiments, R′ independently is an unsubstituted alkyl (e.g., unsubstituted C₁-C₂₀ alkyl, C₁-C₁₅ alkyl, C₁-C₁₀ alkyl, or C₁-C₃ alkyl). In embodiments, R′ independently is unsubstituted C₁-C₃ alkyl. In embodiments, the alkynyl is unsubstituted. In embodiments, the alkynyl is substituted (e.g., with 1, 2, 3, 4, 5, or 6 substituent groups as described herein).

Affixing the suffix “-ene” to a group indicates the group is a divalent moiety. Alkylene refers to a saturated divalent straight or branched chain hydrocarbon group and is exemplified by methylene, ethylene, isopropylene and the like. Likewise, alkenylene refers to an unsaturated divalent straight or branched chain hydrocarbon group having one or more unsaturated carbon-carbon double bonds that may occur in any stable point along the chain, and alkynylene refers to an unsaturated divalent straight or branched chain hydrocarbon group having one or more unsaturated carbon-carbon triple bonds that may occur in any stable point along the chain. In certain embodiments, an alkylene, alkenylene, or alkynylene group may comprise one or more cyclic aliphatic and/or one or more heteroatoms such as oxygen, nitrogen, or sulfur and may optionally be substituted with one or more substituents such as alkyl, halo, alkoxyl, hydroxy, amino, aryl, ether, ester or amide. For example, an alkylene, alkenylene, or alkynylene may be substituted with one or more (e.g., 1, 2, 3, 4, 5, or 6 independently selected substituents) of halogen, —COR′, —CO2H, —CO2R′, —CN, —OH, —OR′, —OCOR′, —OCO2R′, —NH2, —NHR′, —N(R′)2, —SR′ or —SO2R′, wherein each instance of R′ independently is C₁-C₂₀ aliphatic (e.g., C₁-C₂₀ alkyl, C₁-C₁₅ alkyl, C₁-C₁₀ alkyl, or C₁-C₃ alkyl). In embodiments, R′ independently is an unsubstituted alkyl (e.g., unsubstituted C₁-C₂₀ alkyl, C₁-C₁₅ alkyl, C₁-C₁₀ alkyl, or C₁-C₃ alkyl). In embodiments, R′ independently is unsubstituted C₁-C₃ alkyl. In certain embodiments, an alkylene, alkenylene, or alkynylene is unsubstituted. In certain embodiments, an alkylene, alkenylene, or alkynylene does not include any heteroatoms.

Alkenyl: As is used herein, “alkenyl” refers to any linear or branched hydrocarbon chains having one or more unsaturated carbon-carbon double bonds that may occur in any stable point along the chain, e.g. C₂-C₂₀ alkenyl refers to an alkenyl group having 2-20 carbons. For example, an alkenyl group includes prop-2-enyl, but-2-enyl, but enyl, 2-methylprop-2-enyl, hex-2-enyl, hex-5-enyl, 2,3-dimethylbut-2-enyl, and the like. In embodiments, the alkenyl comprises 1, 2, or 3 carbon-carbon double bond. In embodiments, the alkenyl comprises a single carbon-carbon double bond. In embodiments, multiple double bonds (e.g., 2 or 3) are conjugated. An alkenyl group may be unsubstituted or substituted with one or more substituent groups as described herein. For example, an alkenyl group may be substituted with one or more (e.g., 1, 2, 3, 4, 5, or 6 independently selected substituents) of halogen, —COR′, —CO2H, —CO2R′, —CN, —OH, —OR′, —OCOR′, —OCO2R′, —NH2, —NHR′, —N(R′)2, —SR′ or —SO2R′, wherein each instance of R′ independently is C1-C20 aliphatic (e.g., C₁-C₂₀ alkyl, C₁-C₁₅ alkyl, C₁-C₁₀ alkyl, or C₁-C₃ alkyl). In embodiments, R′ independently is an unsubstituted alkyl (e.g., unsubstituted C₁-C₂₀ alkyl, C₁-C₁₅ alkyl, C₁-C₁₀ alkyl, or C₁-C₃ alkyl). In embodiments, R′ independently is unsubstituted C₁-C₃ alkyl. In embodiments, the alkenyl is unsubstituted. In embodiments, the alkenyl is substituted (e.g., with 1, 2, 3, 4, 5, or 6 substituent groups as described herein). In embodiments, an alkenyl group is substituted with a —OH group and may also be referred to herein as a “hydroxyalkenyl” group, where the prefix denotes the —OH group and “alkenyl” is as described herein.

Amino: As used herein, amino refers to groups of the form —N(R′)₂ wherein each R′ is independently selected from hydrogen, alkyl, alkenyl, alkynyl, and aryl as described herein. Alkylamino includes both mono-alkylamino and dialkylamino, unless specified. Mono-alkylamino means an —NH(alkyl) group, in which alkyl is as defined herein. Dialkylamino means an —N(alkyl)₂ group, in which each alkyl may be the same or different and are each as defined herein for alkyl. In embodiments, an alkyl group is a C₁-C₆ alkyl group. The group may be a terminal group or a bridging group. If the group is a terminal group it is bonded to the remainder of the molecule through the nitrogen atom.

Amine: As used herein, amine refers to a group having the amide functional group:

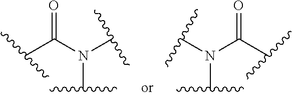 [SMILES: *C(=O)N(*)* ; *C(=O)N(*)*]

An amide group may have 1, 2, or 3 points of attachment to the molecule. Exemplary amide groups include —C(O)N(R′)₂, —C(O)NHR′, —C(O)NH₂, —C(O)NH—, —C(O)NR′—, —NHC(O)—, and —NR′C(O)—, wherein each instance of R′ independently is C₁-C₂₀ aliphatic (e.g., C₁-C₂₀ alkyl, C₁-C₁₅ alkyl, C₁-C₁₀ alkyl, or C₁-C₃ alkyl), or two R′ can combine to form a 3- to 10-membered nitrogen-containing heterocyclyl. In embodiments, R′ independently is an unsubstituted alkyl (e.g., unsubstituted C₁-C₂₀ alkyl, C₁-C₁₅ alkyl, C₁-C₁₀ alkyl, or C₁-C₃ alkyl). In embodiments, R′ independently is unsubstituted C₁-C₃ alkyl. In embodiments, the alkenyl is unsubstituted. In embodiments, the alkenyl is substituted (e.g., with 1, 2, 3, 4, 5, or 6 substituent groups as described herein). In embodiments, an alkenyl group is substituted with a —OH group and may also be referred to herein as a “hydroxyalkenyl” group, where the prefix denotes the —OH group and “alkenyl” is as described herein.

Anhydride linkages: As used herein, anhydride linkages are characterized by two acyl groups joined by an oxygen atom, having the general structure:

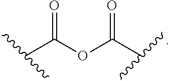 [SMILES: *C(=O)OC(*)=O]

Ester linkage: As used herein, an ester linkage refers to —OC(═O)— or —C(═O)O—; thioester linkage refers to —SC(═O)— or —C(═O)S.

Halogen: As used herein, halogen means fluorine, chlorine, bromine, or iodine.

Heteroalkyl: As used herein, heteroalkyl refers to a branched or unbranched alkyl, alkenyl, or alkynyl group having from 1 to 14 carbon atoms in addition to 1, 2, 3 or 4 heteroatoms independently selected from the group consisting of N, O, S, and P. Heteroalkyls include tertiary amines, secondary amines, ethers, thioethers, amides, thioamides, carbamates, thiocarbamates, hydrazones, imines, phosphodiesters, phosphoramidates, sulfonamides, and disulfides. A heteroalkyl group may optionally include monocyclic, bicyclic, or tricyclic rings, in which each ring desirably has three to six members. Examples of heteroalkyls include polyethers, such as methoxymethyl and ethoxyethyl.

Heteroalkylene: As used herein, heteroalkylene refers to a divalent form of a heteroalkyl group as described herein.

Heterocycle, heterocyclyl, heterocyclic radical, and heterocyclic ring: As used herein, heterocycle, heterocyclyl, heterocyclic radical, and heterocyclic ring are used interchangeably and refer to a stable 3- to 8-membered monocyclic or 7-10-membered bicyclic heterocyclic moiety that is either saturated or partially unsaturated, and having, in addition to carbon atoms, one or more, such as one to four, heteroatoms, as defined above. When used in reference to a ring atom of a heterocycle, the term nitrogen includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or NR+ (as in N-substituted pyrrolidinyl).

### V. METHODS OF USE

a. Treatment

In some embodiments, the formulation described herein is administered to a subject having, suspected of having, or at risk of a disease or disorder associated with the lung. In some embodiments, the formulation described herein is administered to a subject to treat a disease or disorder associated with the lung.

In some embodiments of the present disclosure, the subject has chronic obstructive pulmonary disease. Chronic obstructive pulmonary disease (COPD) (OMIM #606963) is a disorder associated with substantial morbidity and mortality. There are roughly 3,000,000 COPD cases in the United States per year. It is defined by irreversible airflow obstruction due to chronic bronchitis, emphysema, and/or small airways disease. Airflow obstruction is typically determined by reductions in quantitative spirometric indices, including, but not limited to: forced expiratory volume at 1 second (FEV1) and the ratio of FEV1 to forced vital capacity (FVC). Common COPD symptoms include: cough with phlegm, frequent respiratory infections, shortness of breath, wheezing, fatigue, inability to exercise, and chest pressure. Although there is palliative care to mitigate COPD symptoms, there is currently no cure.

In some embodiments of the present disclosure, the subject has asthma. Asthma (OMIM #600807) is a condition in which the airways become inflamed, narrow and swell, and produce excess mucus, all of which makes it difficult for a person suffering from asthma to breathe. There are roughly 3,000,000 asthma cases in the United States per year. Common asthma symptoms include: difficulty breathing, chest pain, cough, wheezing, breathing through the mouth, fast breathing, frequent respiratory infections, shortness of breath at night, chest pressure, flare, anxiety, early awakening, fast heart rate, and throat irritation. Although there is palliative care to mitigate asthma symptoms, there is currently no cure.

In some embodiments of the present disclosure, the subject has pulmonary fibrosis. Pulmonary fibrosis (PF) (OMIM #178500) is a disorder that occurs when lung tissue becomes damaged and scarred. This damaged, scarred tissue makes it difficult to breathe. In some cases, PF can be rapidly progressive and characterized by sequential acute lung injury with subsequent scarring and end-stage lung disease. Common PF symptoms include: shortness of breath, radiographically evident diffuse pulmonary infiltrates, varying degrees of pulmonary inflammation and fibrosis, fatigue, unexplained weight loss, aching muscles and joints, and widening and rounding of the tips of the fingers or toes. Although there is palliative care to mitigate PF symptoms, there is currently no cure.

In some embodiments, the formulation is immune-evading when administered to a subject. In some embodiments, the formulation that does not substantially induce an immune response in the lung, e.g., relative to a control. In some embodiments, an immune-evading formulation does not substantially induce inflammation of the lung tissue. In some embodiments, an immune-evading formulation is not substantially targeted by (i) the mucociliary clearance system, (ii) alveolar macrophages, and/or (iii) monocytes.

b. Target Genes

The formulations described herein comprise oligonucleotides having regions of complementarity to target genes. In some embodiments, the formulation described herein is administered to a subject to treat a disease or disorder associated with the lung by modulating expression of the target gene. In some embodiments, the target genes are over-expressed in a subject having a lung disease or disorder compared with a subject that does not have a lung disease or disorder. In some embodiments, the target genes are under-expressed in a subject having a lung disease or disorder compared with a subject that does not have a lung disease or disorder. In some embodiments, the target genes have mutations that are associated with a lung disease or disorder compared with a subject that does not have mutations in the target gene.

In some embodiments, the target gene is ADORA1. In some embodiments, the target gene is SYK. In some embodiments, the target gene is HRPT1. In some embodiments, a target gene of the present disclosure is a gene associated with chronic obstructive pulmonary disease (COPD), asthma, and/or pulmonary fibrosis. Non-limiting examples of genes associated with COPD include AAT, CHRNA3/5, IREB2, HHIP, FAM13A, and AGER. Non-limiting examples of genes associated with asthma include IL33, TSLP, IL1RL1, ORMDL3, GSDML, HNMT, MUC7, SCGB3A2, ADRB2, HLA-G, TNF, PLA2G7, ALOX5, and CCL11. In some embodiments, a target gene of the present disclosure is a gene associated with asthma and is ADORA1. In some embodiments, a target gene of the present disclosure is a gene associated with asthma and is SYK. Non-limiting examples of genes associated with pulmonary fibrosis include SFTPA2, MUC5B, and TERT.

c. Delivering/Dosing

The formulation of the present disclosure may be in liquid or powdered form. When the formulation is in liquid form, the liquid is composed of droplets. A droplet is a very small drop of a liquid. In some embodiments, the diameter of the droplets is sufficient for pulmonary delivery to lung cells in the subject. Sufficient for pulmonary delivery refers to a droplet size that will likely allow for delivery to pulmonary tissue in a subject. Previous studies have indicated that droplets <5 μm in diameter are likely to be delivered to the lung, droplets between 5-10 μm are likely to be delivered to the lower respiratory tract outside the lung, and droplets >10 μm are likely to be delivered to the upper respiratory tract (Iyer, et al., 2015, Nano-Therapeutics for the Lung: State-of-the-Art and Future Perspectives, Curr Pharm Des., 21(36): 5233-5244).

In some embodiments, liquid formulations of the present disclosure comprises droplets that are <5 μm in diameter. In some embodiments, liquid formulations of the present disclosure comprise droplets that are 5-10 μm in diameter. In some embodiments, liquid formulations comprise droplets that are 0.1-10 μm in diameter. In some embodiments, liquid formulations comprise droplets that are 0.1-5 μm in diameter.

In some embodiments, formulations of the present disclosure may be aerosolized to pulmonarily deliver the formulation to the subject. Aerosolized refers to converting a formulation into a fine spray or suspension that can be inhaled. In some embodiments, aerosolization is accomplished using a propellant to convert liquid or particles into a fine spray or suspension. Non-limiting examples of suitable propellants include compressed air, oxygen, tetrafluoroethane, heptafluoroethane, dimethylfluoropropane, tetrafluoropropane, butane, isobutane, dimethyl ether and other non-CFC and CFC propellants. In some embodiments, a nebulizer is used to aerosolize a formulation for pulmonary delivery. In some embodiments, the nebulizer uses ultrasound waves to generate an aerolized formulation.

In some embodiments, pulmonary delivery of formulations comprising oligonucleotides and lipid nanoparticles takes advantage of the extremely large surface area of alveoli, a dense capillary network, and a relatively thin barrier to absorption that are advantageous when considering the lung versus other routes of delivery (e.g., oral, injection). Non-limiting methods of pulmonary delivery include: nebulization, atomization, intratracheal administration, and intratracheal instillation.

In some embodiments, the formulation is provided to a subject by nebulization. As is used herein, “nebulization: refers to delivery of a formulation in a fine spray or suspension that is inhaled into the lungs by a nebulizer, e.g., a device that use oxygen, compressed air, or ultrasonic power to disperse the particles in a composition into small aerosol droplets that can be directly inhaled. Inhalation from a nebulizer is through a mouthpiece used by the subject.

The efficacy of nebulizing a composition for pulmonary delivery depends on the size of the small aerosol droplets. Generally, the smaller the droplet size, the greater its chance of penetration into and retention in the lung. Large droplets (>10 μm in diameter) are most likely to deposit in the mouth and throat, medium droplets (5-10 μm in diameter) are most likely to deposit between the mouth and airway, and small droplets (<5 μm in diameter) are most likely to deposit and be retained in the lung.

In some embodiments, nebulized formulations are made of droplets that are <5 μm in diameter. In some embodiments, nebulized formulations are made of droplets that are 5-10 μm in diameter. In some embodiments, nebulized formulations are made of droplets that are 0.1-10 μm in diameter. In some embodiments, nebulized formulations are made of droplets that are 0.1-5 μm in diameter.

In one aspect, the disclosure features a method of administering a formulation comprising an oligonucleotide and a lipid nanoparticle to a subject (e.g., a human subject). In one embodiment, the unit dose ranges from about 0.001 mg/kg body weight to 500 mg/kg body weight.

The defined amount can be an amount effective to treat or prevent a disease or disorder, e.g., a disease or disorder associated with low levels of an RNA or protein; a disease or disorder associated with high levels of an RNA or protein; or a disease or disorder associated with expression of a mutant protein.

In one embodiment, a subject is administered an initial dose and one or more maintenance doses of a formulation comprising a stabilizing oligonucleotide and a particle. The maintenance doses may be administered no more than once every 1, 5, 10, or 30 days.

Following treatment, the patient can be monitored for changes in his condition and for alleviation of the symptoms of the disease state. The dosage of the formulation may either be increased in the event the patient does not respond significantly to current dosage levels, or the dose may be decreased if an alleviation of the symptoms of the disease state is observed, if the disease state has been ablated, or if undesired side-effects are observed.

The effective dose can be administered in a single dose or in two or more doses, as desired or considered appropriate under the specific circumstances.

In some cases, a patient is treated with a formulation comprising an oligonucleotide and a lipid nanoparticle in conjunction with other therapeutic modalities.

Certain factors may influence the dosage required to effectively treat a subject, including but not limited to the severity of the disease or disorder, previous treatments, the general health and/or age of the subject, and other diseases present. Moreover, treatment of a subject with a therapeutically effective amount of an oligonucleotide in a formulation can include a single treatment or, preferably, can include a series of treatments. It will also be appreciated that the effective dosage of an oligonucleotide in a formulation used for treatment may increase or decrease over the course of a particular treatment. For example, the subject can be monitored after administering the formulation. Based on information from the monitoring, an additional amount of a formulation comprising a stabilizing oligonucleotide and a particle can be administered.

In some embodiments, the formulation is pulmonarily administered by the subject. In some embodiments, the formulation is pulmonarily administered by a healthcare provider.

### VI. KITS

In certain aspects of the disclosure, kits are provided, comprising a container housing a formulation comprising an oligonucleotide and a lipid nanoparticle. In some embodiments, the individual components of the formulation may be provided in one container. Alternatively, it may be desirable to provide the components of the formulation separately in two or more containers, e.g., one container for oligonucleotide, and at least another for the lipid nanoparticle. The kit may be packaged in a number of different configurations such as one or more containers in a single box. The different components can be combined, e.g., according to instructions provided with the kit. The components can be combined according to a method described herein, e.g., to prepare and administer a pharmaceutical composition. The kit can also include a delivery device.

### VII. EXAMPLES

### Example 1. Design of siRNA Duplex Oligos Targeting HPRT1

Small interfering RNAs (siRNAs) were designed to knockdown the housekeeping gene hypoxanthine phosphoribosyltransferase 1 (HPRT1) (Gene ID: 3251). The HPRT1 protein is a transferase that catalyzes phosphate transfer and plays a central role purine nucleotide generation. The siRNAs were designed as follows:

Transcript selection: Human, cynomolgus monkey (“cyno”), mouse and rat HPRT1 transcripts were obtained from the NCBI RefSeq database. Experimentally validated “NM” transcripts were chosen that contained the maximum number of internal exons: NM_000194.2 for human, NM_001283594.1 for cyno, NM_013556.2 for mouse, and NM_012583.2 for rat. Off-target analysis (below) also utilized the NCBI RefSeq database.

Selection of oligonucleotide sequences: All 18mer sub-sequences and complementary antisense sequences that matched the HPRT1 transcripts in human, cyno, mouse and rat were generated. An A nucleotide was added to the 3′ end of the sense strand, with a complementary U at the 5′ end of the antisense strand, to yield a 19mer duplex. This UA pair was utilized since the antisense (“guide”) strand's 5′-most nucleotide is not exposed to and does not bind target mRNAs when loaded in the RISC complex, and the AGO protein subunit prefers 5′ U nucleotides (Noland and Doudna 2013, Nakanishi 2016). This process yielded 128 candidate 19mer duplexes, which were further evaluated for efficacy characteristics and off-target specificity.

Specificity and efficacy selection: The specificity of the candidate duplexes was evaluated via alignment of both strands to all human and mouse RefSeq transcripts, using the FASTA algorithm with an E value cutoff of 1000. The counts of mismatches between each strand and each transcript (per species) were tabulated. Duplexes were chosen that had at least one 8mer seed (positions 2-9) mismatch on both strands to any human or cyno transcript other than those encoded by the HPRT1 gene, and at least one mismatch at any position on both strands to any mouse or rat transcript other than those encoded by the HPRT1 gene. Seed mismatches are particularly critical for specificity of siRNAs activity (Boudreau et al. 2011). Duplexes were further selected for GC content and thermal asymmetry according to the following guidelines: GC content <55%, >3 Us or As in the antisense 8mer seed, and a G or C at the antisense 19th position. GC content and asymmetry are two important predictors of siRNA efficacy (Akinc, Bettencourt, and Maier 2015). Any duplexes (strands) with homopolymers of 5 or more nucleotides were excluded. Selection according to these parameters yielded 8 duplexes, which were synthesized and screened.

The sequences of two duplex siRNAs that were selected for tested are provided below in Table 1.

TABLE 1
HRPTI siRNA duplex sequences

| | | SEQ |
|---|---|---|
| | | ID |
| Name | Sequence | NO: |
| duplex-H2: | 5′-GGAuAuGcccuuGAcuAuAdTdT-3′ | 1 |
| Sense | | |
| duplex-H2: | 5′-UAuAGUcAAGGGcAuAUCCdTdT-3′ | 2 |
| Antisense | | |
| duplex-H3: | 5′-GAuGAucucucAAcuuuAAdTdT-3′ | 3 |
| Sense | | |
| duplex-H3: | 5′-UuAAAGUUGAGAGAUcAUCdTdT-3′ | 4 |
| Antisense | | |

A lowercase letter denotes a 2′-O-methyl modified ribonucleotide
An uppercase letter denotes a ribonucleotide
dTdT = DNA overhang on the 5′ end and the 3′ end

### Example 2: HRPT1 siRNA Duplexes are Active In Vitro

In order to determine the efficacy of siRNA duplexes of the present disclosure, both HRPT1 duplex H2 and duplex H3 siRNAs knockdown HRPT1 gene expression in vitro. PC9 cells were seeded at a density of 12,500 cells/well in a 96 well plate. HRPT1 duplex H2 and duplex H3 siRNAs ranging from 7.81×10⁻¹²M to 2.5×10⁻⁸ M added to the cells in the presence of transfection reagent (Dharmacon) to transfect the cells.

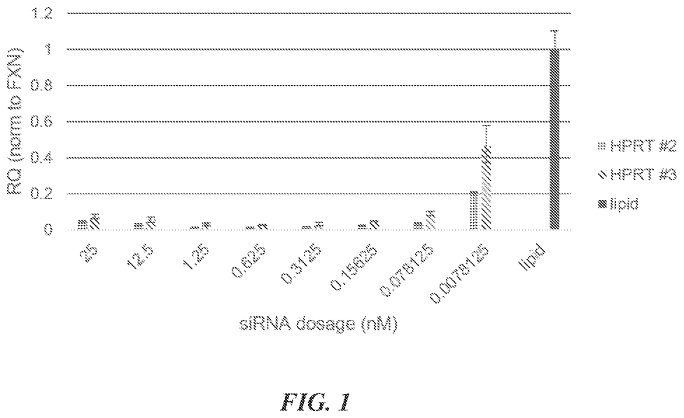

After 48 hours, the cells were collected and lysed. Reverse transcriptase quantitative polymerase chain reaction (RTqPCR) was performed in duplicate or triplicate reactions per sample to measure HRPT1 gene expression. As shown in FIG. 1, both HRPT1 duplex H2 and duplex H3 siRNAs reduced the expression of HRPT1 by up to 90% at 48 hours relative to the control gene FXN.

### Example 3: Lipid Nanoparticle-siRNA Compositions are Active In Vitro

The experiment in Example 2 was repeated with HRPT1 duplex H2 siRNAs either alone or in composition with lipid nanoparticles of the present disclosure comprising the cationic lipid imidazole cholesterol ester (ICE).

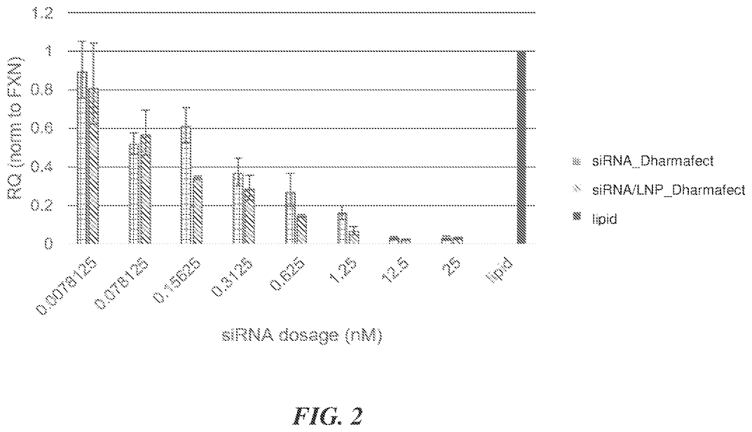

As shown in FIG. 2, compositions comprising HRPT1 duplex H2 siRNAs and lipid nanoparticles (LNPs) are as effective at knocking down gene expression in vitro as HRPT1 duplex H2 siRNAs alone.

### Example 4: HRPT1 Duplex 112 siRNA Knocks Down Gene Expression In Vivo

The ability of HPRT1 duplex H2 siRNA in composition with LNPs to knockdown gene expression in the liver was examined in 6-8 week old CD1 male mice. HPRT1 gene expression was knocked down and measured according to the protocol in Table 2.

TABLE 2
In vivo testing conditions

| | | | | Dosing | Time |
|---|---|---|---|---|---|
| | | Dose | | Route of | points |
| | Test | Levels | No. of | Administra- | (Post |
| Group No. | Article | mg/kg | animals | tion | Dose) |
| Saline | N/A | N/A | 8 | Once on Day | N = 4 (24, |
| AS-02-121- | HPRT1 | 0.1 | 8 | 1 via IV | 72 hr) |
| 01 | siRNA/MC3 | | | injection | |
| AS-02-121- | HPRT1 | 0.25 | 8 | | |
| 01 | siRNA/MC3 | | | | |
| AS-02-121- | HPRT1 | 0.5 | 8 | | |
| 01 | siRNA/MC3 | | | | |
| AS-02-121- | HPRT1 | 0.1 | 8 | | |
| 02 | siRNA/C12- | | | | |
| | 200 | | | | |
| AS-02-121- | HPRT1- | 0.25 | 8 | | |
| 2 | siRNA/C12- | | | | |
| | 200 | | | | |
| AS-02-121- | HPRT1 | 0.5 | 8 | | |
| 02 | siRNA/C12- | | | | |
| | 200 | | | | |

AS-02 = HPRT1 duplex H2 siRNA
MC3 = (6Z,9Z,28Z,31Z)-heptatriconta-6,9,28,31-tetraen-19-yl 4-(dimethylamino)butanoate

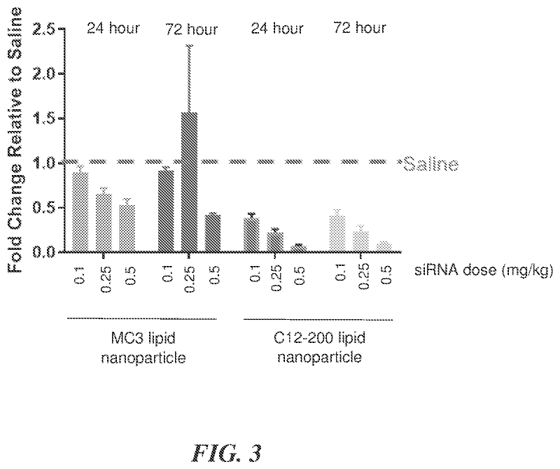

As shown in FIG. 3, administration of 0.5 mg/kg HPRT1 duplex H2 siRNAs knocked-down HPRT1 gene expression at 72 hours when in composition with either MC3 or C12-200 lipid nanoparticles. Notably, C12-200 LNPs in composition with duplex H2 siRNA knocked-down HPRT1 gene expression by at least two-fold regardless of the dosage levels of siRNA or the time points post-dose.

### Example 5: HPRT1 Duplex 112 siRNAs Knockdown Gene Expression in the Lung

The ability of HPRT1 duplex H2 siRNAs in composition with LNPs to knockdown gene expression in the lungs was examined in vivo. HPRT1 gene expression was knocked down and measured according to the protocol in Table 3. The compositions comprising lipid nanoparticles and HPRT1 siRNA duplex were administered to the lungs of CD1 mice by nebulization.

TABLE 3
In vivo testing conditions

| | | # of | | | |
|---|---|---|---|---|---|
| Group | Test | Expo- | Length of | | N per |
| No. | Article | sures | Exposure | Termination | group |
| 1 | Saline | Single | 3 hours/ | 24 hrs post last dose | 8 (n = 4 |
| | | | exposure @ | 72 hrs post last dose | per time |
| 2 | HPRT1 | Single | 0.6 mg/mL | 24 hrs post last dose | point) |
| | H2- | | | 72 hrs post last dose | |
| 3 | siRNA | Double | | 24 hrs post last dose | |
| | | | | 72 hrs post last dose | |

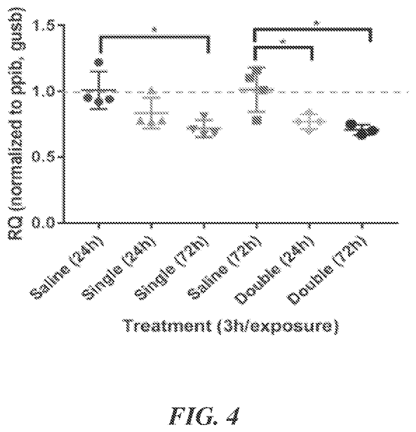

As shown in FIG. 4, HPRT1 duplex H2 siRNAs knockdown HPRT1 mRNA expression in the lungs of mice by about 30%. Importantly, compositions comprising LNPs and siRNAs delivered to the lungs did not cause gross anatomical adverse effects in the mice (data not shown).

The LNP formulation is continuing to be optimized for even more improved siRNA delivery to the lung.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present disclosure.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, e.g., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, e.g., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (e.g. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, e.g., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

What is claimed is:

1. A method of delivering an oligonucleotide to lung cells of a subject, the method comprising pulmonarily administering to the subject an aerosolized formulation that comprises i) an oligonucleotide comprising an antisense strand having a length of 8-30 nucleotides and having a region of complementarity with a target gene in the lung cells, wherein the oligonucleotide comprises at least one modified nucleotide, and ii) a lipid nanoparticle.

2. The method of claim 1, wherein the lipid nanoparticle comprises one or more cationic lipids, one or more non-cationic lipids, one or more PEG-modified lipids, or a combination thereof.

3. The method of claim 1 or claim 2, wherein the formulation is an immune-evading formulation.

4. The method of any one of claims 1-3, wherein the one or more PEG-modified lipids are conjugated lipids that inhibits aggregation of particles.

5. The method of any one of claims 1-4, wherein the lipid nanoparticle comprises a cationic lipid selected from DOTAP (1,2-dioleyl-3-trimethylammonium propane), DODAP (1,2-dioleyl-3-dimethylammonium propane), DOTMA (N-[1-(2,3-dioleyloxy)propyl]-N,N,N-trimethylammonium chloride), DLinKC2DMA, DLin-KC2-DM, C12-200, cKK-E12 (3,6-bis(4-(bis(2-hydroxydodecyl)amino)butyl)piperazine-2,5-dione), HGT5000, HGT5001, HGT4003, ICE, OF-02 and combinations thereof.

6. The method of any one of claims 1-5, wherein the lipid nanoparticle comprises a non-cationic lipid selected from DSPC (1,2-distearoyl-sn-glycero-3-phosphocholine), DPPC (1,2-dipalmitoyl-sn-glycero-3-phosphocholine), DOPE (1,2-dioleyl-sn-glycero phosphoethanolamine), DOPC (1,2-dioleyl-sn-glycero-3-phosphotidylcholine) DPPE (1,2-dipalmitoyl-sn-glycero-3-phosphoethanolamine), DMPE (1,2-dimyristoyl-sn-glycero phosphoethanolamine), DOPG (1,2-dioleoyl-sn-glycero-3-phospho-(1′-rac-glycerol)) or combinations thereof.

7. The method of any one of claims 1-6, wherein the lipid nanoparticle comprises ICE, DOPE and DMG-PEG2K.

8. The method of any one of claims 1-7, wherein the cationic lipid constitutes about 30-80% of the lipid nanoparticles by molar ratio.

9. The method of any one of claims 1-8, wherein the ratio of cationic lipids:non-cationic lipids:PEGylated lipids is approximately 60:45:5 by molar ratio.

10. The method of any one of claims 1-9, wherein the lipid nanoparticle has a size of about 80 nm to 120 nm as measured by dynamic light scattering.

11. The method of any one of claims 1-10, wherein the oligonucleotide is encapsulated in the lipid nanoparticle.

12. The method of any one of claims 1-11, wherein providing the formulation to the lung of the subject is by nebulization.

13. The method of any one of claims 1-12, wherein the lung cells are lung epithelial cells.

14. The method of any one of claims 1-13, wherein the subject is identified as being at risk of lung disease, e.g., COPD, asthma, or pulmonary fibrosis.

15. The method of any one of claims 1-14, wherein the region of complementarity comprises at least 15 contiguous nucleotides of the target gene.

16. The method of any one of claims 1-15, wherein the oligonucleotide is single stranded.

17. The method of any one of claims 1-16, wherein the oligonucleotide further comprises a sense strand, and wherein the antisense strand and the sense strand form a duplex region.

18. The method of claim 17, wherein the sense strand and/or the antisense strand is 15-25 nucleotides in length.

19. The method of claim 17 or 18, wherein the region of complementarity is 19 nucleotides in length.

20. The method of any one of claims 17-19, wherein the oligonucleotide comprising the sense strand and the antisense strand and having a duplex region further comprises a single-stranded overhang on the sense and/or antisense strand in the range of 1 to 2 nucleotides in length.

21. The method of claim 20, wherein the sense strand and/or the antisense strand have a 3′ overhang comprising two deoxythymidines.

22. The method of any one of claims 17-21, wherein the oligonucleotide comprises one blunt end.

23. The method of any one of claims 17-22, wherein the nucleotide at the 5′ end of the sense strand and/or antisense strand is uracil.

24. The method of any one of claims 1-23, wherein the modified nucleotide comprises a 2′-fluoro or a 2′-O-methyl.

25. The method of any one of claims 17-24, wherein the oligonucleotide comprises 2′-O-methyl modified nucleotides in both the sense strand and the antisense strand.

26. The method of claim 25, wherein the oligonucleotide comprises more 2′-O-methyl modified nucleotides on the sense strand than on the antisense strand.

27. A formulation comprising:
an oligonucleotide having at least one modified nucleotide and comprising an antisense strand of 8-30 nucleotides in length; and
a lipid nanoparticle;
wherein the antisense strand has a region of complementarity with a target gene, and wherein the composition is formulated for delivery to the lung.

28. The formulation of claim 27, wherein the lipid nanoparticle comprises ICE, DOPE and DMG-PEG2K.

29. The formulation of claim 27 or 28, wherein the ratio of ICE:DOPE:DMG-PEG2K is approximately 60:45:5 by molar ratio.

30. The formulation of any one of claims 27-29, wherein the lipid nanoparticle has a size of about 80 nm to 120 nm as measured by dynamic light scattering.

31. The formulation of any one of claims 27-30, wherein the oligonucleotide is encapsulated in the lipid nanoparticle.

32. The formulation of any one of claims 27-31, wherein the oligonucleotide is single stranded.

33. The formulation of any one of claims 27-31, wherein the oligonucleotide further comprises a sense strand, and wherein the antisense strand and the sense strand form a duplex region.

34. The formulation of claim 33, wherein the sense strand and/or the antisense strand is 15-25 nucleotides in length.

35. The formulation of claim 33 or 34, wherein the oligonucleotide comprising the sense strand and the antisense strand and having a duplex region further comprises a single-stranded overhang on the sense and/or antisense strand in the range of 1 to 2 nucleotides in length.

36. A method of delivering a formulation to lung cells comprising administering the formulation of any one of claims 27-35 to the lung of the subject by nebulization.

37. The method of claim 36, wherein the lung cells are lung epithelial cells.

38. The method of claim 36 or 37, wherein the subject has, is at risk of having, or is suspected of having a lung disease, e.g., COPD, asthma, or pulmonary fibrosis.

39. A kit comprising a container housing the formulation of any one of claims 27-35.
